(19) United States
(12) Patent Application Publication
(10) Pub. No.: US 2009/0220611 A1
(43) Pub. Date: Sep. 3, 2009

(54) Microparticles With Modified Release of At Least One Active Principle and Oral Pharmaceutical Form Comprising Same

(76) Inventors: Frederic Dargelas, Pessac (FR); Florence Guimberteau, Montussan (FR); Catherine Castan, Orlienas (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(21) Appl. No.: 11/992,769

(22) PCT Filed: Sep. 27, 2006
(86) PCT No.: PCT/FR2006/050944

§ 371 Date: Mar. 20, 2009

(30) Foreign Application Priority Data
Sep. 30, 2005 (FR) 0552985

(51) Int. Cl. A61K 9/16

(52) U.S. Cl. 424/495; 424/497; 424/494

(57) ABSTRACT

The invention concerns microparticulate systems with modified release of oral active principle(s). The invention aims at providing a novel pharmaceutical with time-dependent and pH-dependent release mechanism, enabling: a) the latent period preceding the release of the active principle in the stomach; b) the pH triggering the release of the active principle in the intestine; c) the release speed of the active principle. This is achieved through the use of coated microparticles made from particles of active principle each coated with two coating films A and B. A comprises: film-forming (co)polymer (A1) insoluble in fluids of the gastrointestinal tract; ethylcellulose (co)polymer (A2) soluble in fluids of the gastrointestinal tract; plasticizing polyvinylpyrrolidone (A3); castor oil/optionally a surfactant and/or magnesium stearate lubricant (A4). B comprises a hydrophilic polymer (B1) bearing ionized groups with neutral pH (EUDRAGIT® L100-55) and a hydrophobic compound (B2) (LUBRITAB®). The invention also concerns medicines based on said microparticles.

### FIELD OF THE INVENTION

The field of the present invention is that of micro-particulate systems with modified release, for example delayed, sustained and/or pulsed release, of active principle(s) (AP, denoting one or more active principles), more especially intended for oral administration.

The APs envisioned in the present invention are especially pharmaceutical APs, and in particular those that are mainly absorbed in the small intestine. The invention is firstly directed toward discrete coated microparticles that each govern, independently but in an overall homogeneous manner, the modified, i.e. especially delayed and sustained, in vivo release of the AP they contain.

Taken together, these microparticles may be included in the constitution of dry pharmaceutical forms such as tablets, powder sachets, powders for suspension to be reconstituted or gel capsules, or of liquid pharmaceutical forms such as syrups or suspensions.

### CONTEXT OF THE INVENTION

One of the essential aims of modified-release pharmaceutical forms, whether they are sustained-release forms or delayed-release or pulsed-release forms or combinations of these modified-release forms with immediate-release forms, is to sustain the duration of action during which the plasma concentration of AP is higher than the minimum level of therapeutic efficacy, while at the same time maintaining the bioavailability of the AP at the highest possible level.

A second essential aim of modified-release pharmaceutical forms is to ensure that the active principle will be effectively released. This point is particularly important in the case of antidiabetic or antihypertensive agents for which the bioabsorption of the AP is vital for the patient.

The plasma concentration profile of an AP generally comprises a first phase during which the concentration rises to its maximum. It is followed by a second phase, during which the concentration decreases, for example mono- or bi-exponentially.

The first phase generally corresponds to the phase of bioabsorption of the active principle. The second phase corresponds to the phase of distribution and elimination of the active principle. The kinetic constants describing the second phase of distribution and elimination are set by the nature of the active principle.

In order to prolong the duration of action of the AP, it is thus possible to vary essentially the first phase, i.e. the bioabsorption of the active principle. Two mutually non-exclusive strategies may be envisioned.

The first strategy consists in increasing the AP bioabsorption time, so as to lengthen the absorption phase and thus to prolong the time for which the plasma concentration is at a maximum. In concrete terms, this amounts to spreading the plasma concentration profile over an increased time.

The second strategy consists in delaying the plasma concentration peak while at the same time keeping it as high as possible. In this way, the decrease phase (AP distribution/elimination) occurs as late as possible and the AP action time is thus increased, particularly when the modified-release form is used in conjunction with an immediate-release form that ensures therapeutic cover in the first moments after administration.

In the two strategies, the bioavailability of the AP should be maintained at a high level. This may be evaluated by measuring the area under the curve of the plasma concentration of the AP as a function of time.

In order to prolong the action time of an AP, the sustained-release forms release the AP slowly and continuously, over a period of several hours, for example 8 hours. This slow and continuous release allows the AP to be absorbed over a longer period, on condition that the AP resides in its bioabsorption window for a sufficient time. However, many APs have a narrow bioabsorption window located in the upper parts of the small intestine (duodenum and jejunum). In this case, the residence time of the AP in its bioabsorption window is limited, to the extent that for such sustained-release forms, a large part of the AP is released outside the bioabsorption window and is not absorbed. The plasma concentration of AP remains low (low bioavailability) and the duration of action is not increased.

Another type of modified-release form is constituted by enteric delayed-release forms. The AP is not released as long as the form remains in the stomach at acidic pH. On the other hand, the release is rapid as soon as the pH rises, generally when the form enters the small intestine. Such enteric pharmaceutical forms are termed “pH-dependent”. The AP is rapidly released in its bioabsorption window. The bioavailability may then be high, but to the detriment of the bioabsorption duration. In addition, the plasma concentration peak is early, particularly when the gastric emptying is rapid, which is the case in the fasted state. As a result, such pharmaceutical forms have a limited duration of action.

A second drawback of enteric forms arises from the large variability of duration of the gastric emptying within the same individual and between two different individuals. This leads to very great inter- and intra-individual variability of the plasma concentration profiles of the AP. This variability is unacceptable for APs such as antihypertensive or antidiabetic agents, since it puts the patients at risk. For these patients, it is in fact vital for the AP to be effectively released at the desired moment, even in the case of abnormally late gastric emptying.

To overcome these insufficiencies, patent application WO-A-03/030 878 proposed a modified-release form of AP according to a pH-dependent and time-dependent twofold mechanism. The time-dependent release is triggered after a predetermined residence time in the stomach. The pH-dependent release takes place under the effect of a rise in pH, when the pharmaceutical form passes from the stomach to the intestine.

These two release-triggering factors placed in parallel give the pharmaceutical formulation great safety of use. The sustained release of the AP is thus ensured after a predetermined lag time, even if the variation in pH has not intervened as a triggering factor, i.e. even if the pharmaceutical form has not left the stomach to enter the intestine. This pharmaceutical form represents a considerable progress over enteric forms since it ensures that the AP will be released even in the case of abnormally long gastric retention.

This form according to WO-A-03/030 878 is, however, improvable since, in order to increase the duration of action, it would be very useful to be able to extend the bioabsorption period. To this end, it would be practical to have a pharmaceutical form with a twofold mechanism of release (pH and time) for which a) the lag time in the stomach, b) the pH triggering release in the small intestine and c) the rate of release of the AP in the intestine, are adjustable as a function of the nature of the AP, of the extent of its bioabsorption window and of its administration conditions.

If we consider, for example, the case of an AP administered in the fed state and having a narrow bioabsorption window, the gastric emptying will be prolonged, but the duration of passage before the bioabsorption window will be relatively short. It would thus be practical for the lag time in the stomach to be relatively long, for the release to take place as soon as the pharmaceutical form enters the intestine and, finally, for this release to be relatively rapid so as to release the AP in its bioabsorption window.

On the contrary, in the case of an AP administered in the fasting state and having a bioabsorption window extended over the entire small intestine, the gastric emptying will be rapid, but the time of passage in the bioabsorption window will be relatively long. It would then be practical for the lag time in the stomach to be short and for the release of the AP to be triggered relatively late in the intestine and/or to be sustained.

There is thus a need for a pharmaceutical form with a twofold mechanism of release (pH and time) for which it is possible to adjust, conveniently and independently of each other, the following three parameters:
a) the lag time in the stomach,
b) the pH that triggers the release of the AP in the intestine,
c) the rate of release of the AP in the stomach and/or in the intestine.

This would make it possible especially to optimize the delivery of the AP as a function of its absorption window, with the consequences of optimizing the absorption and thus the duration of action of the AP, of limiting the side effects and in certain cases the doses, and of improving the comfort of the patients and the compliance with the treatment by limiting the number of dosage intakes.

### OBJECTIVES OF THE INVENTION

In such a state of the art, one of the essential objectives of the present invention is to provide a novel multi-microparticulate pharmaceutical formulation for the oral administration of APs absorbed in the gastrointestinal tract at least in the upper parts of the gastrointestinal tract, which makes it possible to increase the time for which the plasma concentration of AP is greater than or equal to the minimum plasma concentration for therapeutic efficacy.

Another object of the present invention is to propose a system that ensures the modified release of the AP, by means of a twofold mechanism of “time-dependent” and “pH-dependent” release. These two factors triggering the release of AP, placed in parallel, ensure the release of the AP after a predetermined lag time, even if the variation in pH has not taken place as a triggering factor.

Another object of the present invention is to provide a novel multi-microparticulate pharmaceutical formulation for the administration of AP, which makes it possible to adjust, independently of each other, the following three parameters:
a) the lag time preceding the release of the AP in the stomach, even up to the point of eliminating this lag time,
b) the pH that triggers the release of the AP in the intestine,
c) the rate of release of the AP in the stomach and/or in the intestine.

Another essential object of the present invention is to provide a multi-microparticulate pharmaceutical formulation that ensures the release of the AP despite the inter- and intra-individual variability of gastric emptying.

Another object of the present invention is to propose a pharmaceutical form at least partly formed from a plurality of coated microparticles avoiding the use of large amounts of coating.

Another object of the present invention is to propose a pharmaceutical form comprising a plurality of coated microparticles making it possible to present the AP in a form that is easy to swallow: sachet, suspension or orodispersible tablet, for example.

Another object of the invention is to propose a pharmaceutical form formed at least partly from a plurality of coated microparticles, allowing several different APs to be mixed together.

Another object of the present invention is to propose a pharmaceutical form formed at least partly from a plurality of coated microparticles having different lag times and/or different rates of release.

### BRIEF DESCRIPTION OF THE INVENTION

Having set themselves the above objectives, inter alia, the inventors have, to their credit, developed a multi-microparticulate delayed-release and sustained-release pharmaceutical form, with “time-dependent” release and “pH-dependent” release, with independently adjustable lag time and release time, and individually covered with at least two coating films allowing the delayed and sustained release of the AP.

The inventors have also, to their credit, conceived an adapted strategy for achieving the objects they set themselves. This strategy is the following:
1. an excessively large amount of AP or all of the AP should not be released early into the stomach, so as to be able to prolong the bioabsorption time;
2. the pH that triggers the release of the AP in the intestine should be adjustable;
3. the AP should be able to be released in the intestine, gradually and at an adjustable rate.

Thus, the invention relates firstly to coated “reservoir” microparticles containing at least one active principle (AP) and being of the type:
constituted by AP particles each covered with at least two different coating films (i.e. of different composition),
with a mean diameter of less than 2000 microns, preferably between 50 and 800 microns and even more preferentially between 100 and 600 microns;
   characterized in that, in combination, these coating films are capable:
of ensuring a release of the AP governed by two different triggering mechanisms, one based on a variation in pH and the other allowing the release of the AP after a predetermined residence time in the stomach,
of inducing in vitro dissolution behavior (performed in a paddle dissolutest in accordance with the European Pharmacopeia 5.2 or a device of type II in US Pharmacopeia 28-NF 23, maintained at 37° C. and rotated at 100 rpm) such that:
   at constant pH 1.4, the dissolution profile comprises a lag phase of adjustable duration of less than or equal to 8 hours, preferably less than or equal to 5 hours and even more preferentially between 1 and 5 hours;
   the passage from pH 1.4 to pH 7.1 leads to a sustained-release phase of adjustable duration, starting without a lag time and such that t½ is between 0.25 and 20 hours, preferably between 0.25 and 12 hours, more preferably between 0.25 and 8 hours and even more preferentially between 0.25 and 4 hours, in which t½ is the time required to release 50% of at least one of the active principles contained in the coated microparticles.

More specifically, the invention relates to “reservoir” microparticles containing at least one active principle (AP) and being of the type:
constituted by AP particles each covered with at least two different coating films,
with a mean diameter of less than 2000 microns, preferably between 50 and 800 microns and even more preferentially between 100 and 600 microns;
   characterized in that each microparticle comprises:
at least one coating film (A) having the following composition:
   (A1) at least one film-forming (co)polymer (A1) that is insoluble in the liquids of the gastrointestinal tract;
   (A2) at least one (co)polymer (A2) that is soluble in the liquids of the gastrointestinal tract;
   (A3) at least one plasticizer (A3);
   (A4) optionally at least one surfactant and/or lubricant (A4);
and at least one coating film (B) constituted of a composite material comprising at least one hydrophilic polymer (B1) bearing groups that are ionized at neutral pH and at least one hydrophobic compound (B2).

The combination of the two types of coating film A and B makes it possible:
to ensure release of the AP governed by two different triggering mechanisms, one based on a variation in pH and the other allowing the release of the AP after a predetermined residence time in the stomach,
to induce in vitro dissolution behavior (performed in a paddle dissolutest in accordance with the European Pharmacopeia 5.2 or a device of type II in US Pharmacopeia 28-NF23, maintained at 37° C. and rotated at 100 rpm) such that:
   at constant pH 1.4, the dissolution profile comprises a lag phase of adjustable duration of less than or equal to 8 hours, preferably less than or equal to 5 hours and even more preferentially between 1 and 5 hours;
   the passage from pH 1.4 to pH 7.1 leads to a sustained-release phase of adjustable duration, starting without a lag time and such that t½ is between 0.25 and 20 hours, preferably between 0.25 and 12 hours, more preferably between 0.25 and 8 hours and even more preferentially between 0.25 and 4 hours, in which t½ is the time required to release 50% of at least one of the active principles contained in the coated microparticles.

One characteristic of the microparticles according to the invention is that the release of the AP is delayed and sustained in a controlled manner.

Advantageously, the microparticles according to the invention are covered with two coating films, a film A and a film B. Film A may be either the inner layer or the outer layer. According to one preferred mode, film A is the inner layer and film B is the outer layer.

The Applicant has, to its credit, developed, entirely surprisingly and unexpectedly, such a pharmaceutical formulation that combines, in microparticles with modified release of AP, two layers for controlling the release of the AP, in order to achieve the above-targeted objectives.

This was all the less foreseeable since it might have been feared that physical mixing of the layers A and B would take place, due to the use during the coating operation of solvents that are common to the two layers. Such mixing is liable to disrupt and render uncontrollable the release of the AP from these microparticles.

The Applicant has also, to its credit, developed a system that combines two layers, which achieves the above-targeted objectives without, however, requiring prohibitive coating thicknesses that would entail a reduction in the AP content of the microparticles, an increase in the preparation times and the amounts of product used, and thus, an increase in the cost.

### DETAILED DESCRIPTION OF THE INVENTION

In the description of the invention, use is made of the term “coated microparticles” to denote AP microparticles coated with at least one coating that allows modified release of AP. Uncoated AP microparticles (i.e. before coating) may be, for example, neutral cores covered with at least one layer containing AP, or pure AP microparticles, or alternatively granules formed by a matrix of support excipients including the AP. The term “microparticles” will cover both coated microparticles according to the invention and uncoated microparticles.

These coated microparticles may be likened to vehicles allowing the transportation and release of at least one AP and possibly of one or more other active principles, in the small intestine or even in the large intestine.

The microparticle diameters under consideration in the present description are, unless otherwise indicated, volume-average diameters.

It is particularly advantageous to be able to give or not give the modified-release microparticles, especially in the case of AP whose absorption occurs mainly in the upper parts of the gastrointestinal tract, a lag period of adjustable duration. During this lag period, there is no or virtually no release of AP, by virtue of the leaktight barrier formed by the outer coating film. This may make it possible to make the in vivo release coincide with the passage in the absorption window that is specific to a given AP.

Another unique advantage of such a system is that of being able to obtain, by mixing with an immediate-release pharmaceutical form or microparticles, or alternatively by mixing with another pharmaceutical form or microparticles with modified release of AP, release profiles having several waves of release of AP (one single or several identical or different APs) or ensuring, via adequate adjustment of the various fractions, a constant plasma concentration level of the AP.

The triggering pH, and thus the moment of release of the AP in the intestine, is adjusted by means of a suitable formulation of hydrophilic polymer(s) bearing groups that are ionized at neutral pH B1 and of hydrophobic compound(s) B2 and as a function of the weight ratio (B2)/(B1).

Commercially, (co)polymers exist (of (meth)acrylic acid or of cellulose phthalates, for example), which cause triggering of the release at a pH that may range from 5 to 7. When the triggering pH is 5, the release takes place immediately on leaving the stomach, at the start of the intestine (in the duodenum). When the triggering pH increases, the release takes place later and later after passage into the intestine.

One of the determining advantages of the multimicroparticulate pharmaceutical formulation, with delayed and controlled release of AP, according to the invention is that of effecting the in vivo intervention of two factors triggering the release of the AP in the gastrointestinal tract, namely:
the residence time in the stomach: “time-dependent” release,
the variation in pH: “pH-dependent” release.

These two factors triggering the release of AP act in parallel, and as such give the pharmaceutical formulation great safety of use. The release of the AP is thus ensured after a preset lag time, even if the variation in pH has not taken place as a triggering factor. The problems of inter-individual variability (especially of gastric emptying) are thus overcome. The therapeutic efficacy of the medicament comprising such a pharmaceutical formulation is ensured, while respecting predefined times adapted to the targeted therapeutic performance.

Moreover, the rate of release is adjusted, for example, in the following manner:
by controlling the thickness of the coating A;
via the weight ratios between the components A1, A2, A3 and possibly A4, of the coating layer A. According to one preferred characteristic of the coated microparticles in accordance with the invention, the coating film A has a coating ratio (Tp_{A})—expressed as a dry weight % relative to the total mass of the coated microparticles—such that TpA≧2%, preferably TpA≧3% and even more preferentially TpA≧4%.

This characteristic corresponds to a threshold thickness for the layer A, below which its mechanical strength and its release-modifying function are no longer ensured.

According to one preferred characteristic of the coated microparticles in accordance with the invention, the coating film A has a coating ratio (Tp_{A})—expressed as a dry weignt % relative to the total mass of the coated microparticles—of less than or equal to 50%.

According to one preferred embodiment of the invention, as regards the coating film A:
(A1) is chosen from the group comprising:
   water-insoluble cellulose derivatives, preferably ethylcellulose and/or cellulose acetate,
   acrylic derivatives, for example copolymers of (meth)acrylic acid and of alkyl (e.g. methyl) ester, copolymers of acrylic and methacrylic acid ester bearing at least one quaternary ammonium group (preferably at least one copolymer of alkyl (meth)acrylate and of trimethylammonioethyl methacrylate chloride) and more specifically the products sold under the brand names EUDRAGIT® RSand/or RL,
   polyvinyl acetates,
   and mixtures thereof;
(A2) is chosen from the group comprising:
   nitrogenous (co)polymers, preferably from the group comprising polyacrylamides, poly-N-vinylamides, polyvinylpyrrolidones (PVP) and poly-N-vinyllactams;
   water-soluble cellulose derivatives,
   polyvinyl alcohols (PVA),
   polyoxyethylenes (POE),
   polyethylene glycols (PEG),
   and mixtures thereof;
   polyvinylpyrrolidone being particularly preferred;
(A3) is chosen from the group comprising:
   cetyl alcohol esters
   glycerol and esters thereof, preferably from the following subgroup: acetylated glycerides, glyceryl monostearate, glyceryl triacetate, glyceryl tributyrate,
   phthalates, preferably from the following subgroup: dibutyl phthalate, diethyl phthalate, dimethyl phthalate, dioctyl phthalate,
   citrates, preferably from the following subgroup: acetyl tributyl citrate, acetyl triethyl citrate, tributyl citrate, triethyl citrate,
   sebacates, preferably from the following subgroup: diethyl sebacate, dibutyl sebacate,
   adipates,
   azelates,
   benzoates,
   plant oils,
   fumarates, preferably diethyl fumarate,
   malates, preferably diethyl malate,
   oxalates, preferably diethyl oxalate,
   succinates; preferably dibutyl succinate,
   butyrates,
   cetyl alcohol esters,
   salicylic acid,
   triacetin,
   malonates, preferably diethyl malonate,
   castor oil (this being particularly preferred),
   and mixtures thereof;
(A4) is chosen from the group comprising:
   anionic surfactants, preferably from the subgroup of alkali metal or alkaline-earth metal salts of fatty acids, stearic acid and/or oleic acid being preferred,
   and/or nonionic surfactants, preferably from the following subgroup:
      polyoxyethylenated oils, preferably hydrogenated polyoxyethylenated castor oil,
      polyoxyethylene-polyoxypropylene copolymers,
      polyoxyethylenated sorbitan esters,
      polyoxyethylenated castor oil derivatives,
      stearates, preferably calcium, magnesium, aluminum or zinc stearate,
      stearylfumarates, preferably sodium stearylfumarate,
      glyceryl behenates,
      and mixtures thereof.

According to this preferred embodiment of the invention, and as regards the coating film B:
B has a coating ratio (Tp_{B})—expressed as a dry weight % relative to the total mass of the coated
microparticles—of less than or equal to 50%;
the weight ratio (B2)/(B1) is between 0.2 and 1.5 and preferably between 0.45 and 1.0,
the hydrophobic compound (B2) is selected from products that are crystalline in the solid state and that have a melting point Tm(B2)≧40° C., preferably Tm(B2)≧50° C. and even more preferentially 50° C.≦Tm(B2)≦90° C.

Advantageously, the hydrophilic polymer bearing groups that are ionized at neutral pH (B1) is chosen from the group comprising:
B1.a copolymers of (meth)acrylic acid and of alkyl (e.g. methyl) ester of (meth)acrylic acid (for example EUDRAGIT® S or L);
B1.b cellulose derivatives, preferably: cellulose acetates, cellulose phthalates, cellulose succinates and even more preferentially hydroxypropylmethylcellulose phthalates, hydroxypropylmethylcellulose acetates and hydroxypropylmethylcellulose succinates;
and mixtures thereof.

The preferred polymers B1 are copolymers of (meth)acrylic acid and of alkyl (e.g. C1-C6 alkyl) esters of (meth)acrylic acid. These copolymers are, for example, of the type such as those sold by the company Röhm Pharma Polymers under the brand name EUDRAGIT®, of L and S series (for instance EUDRAGIT® L100, S100, L30 D-55 and L100-55). These copolymers are anionic copolymers that are soluble in aqueous medium at pH values above those encountered in the stomach.

Advantageously, compound B2 is chosen from the following group of products:
B2.a plant waxes taken alone or as mutual mixtures;
B2.b hydrogenated plant oils taken alone or as mutual mixtures;
B2.c mono- and/or di- and/or triesters of glycerol and of at least one fatty acid;
B2.d mixtures of monoesters, diesters and triesters of glycerol and of at least one fatty acid;
B2.e and mixtures thereof.

Preferably, compound B2 is chosen from the following group of products: hydrogenated cottonseed oil, hydrogenated soybean oil, hydrogenated palm oil, glyceryl behenate, hydrogenated castor oil, tristearine, tripalmitine, trimyristine, yellow wax, hard fat or fat useful as suppository bases, anhydrous dairy fat, lanolin, glyceryl palmitostearate, glyceryl stearate, lauryl macrogolglycerides, cetyl alcohol, polyglyceryl diisostearate, diethylene glycol monostearate, ethylene glycol monostearate, omega-3 and any mixture thereof.

Better still, compound B2 is chosen from the following subgroup of products: hydrogenated cottonseed oil, hydrogenated soybean oil, hydrogenated palm oil, glyceryl behenate, hydrogenated castor oil, tristearine, tripalmitine, trimyristine and any mixture thereof.

In practice, and without this being limiting, it is preferable for compound B2 to be chosen:
from the group of products sold under the following brand names: Dynasan®, Cutina®, Hydrobase®, Dub@, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol Diisostearique®, Geleol®, Hydrine®, Monthyle®, and mixtures thereof;
and also from the group of additives whose codes are as follows: E 901, E 907, E 903, and mixtures thereof;
and, preferably, from the group of products sold under the following brand names: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

In addition, for the APs under consideration in the present invention, whose absorption window is limited to the upper parts of the gastrointestinal tract, it is particularly advantageous for the delayed- and then sustained-release form to be a plurality of coated microparticles. Specifically, for such a form, the dose of AP to be administered is distributed between a large number of coated microparticles (typically 10 000 for a 500 mg dose) and as a result has the following intrinsic advantages:
The residence time of the coated microparticles in the upper parts of the gastrointestinal tract may be prolonged, which ensures an increase in the duration of passage of the AP in the absorption windows and thus maximizes the bioavailability of the AP.
The use of a mixture of coated microparticles with different delayed- and controlled-release profiles makes it possible to produce release profiles having several waves of release or ensuring, via adequate adjustment of the various fractions, a constant plasma concentration level of the AP.
The variability of gastric emptying is smaller, since the emptying that takes place here over a large number of particles is statistically more reproducible.
Contact of tissues with a high dose of AP (the problem of “dose dumping”) is avoided. Specifically, each microparticle contains only a very small dose of AP. This thus circumvents the risk of deterioration of tissues by local over-concentration of a corrosive AP.
It is possible to present these microparticles in sachet, gel capsule or tablet form. When the dose of AP is high (500 mg or more), monolithic forms are too large in size to be swallowed easily. It is then particularly advantageous to have available a microparticulate form that ensures the delayed and controlled release of the AP that a person skilled in the art can form into disintegrable tablets or sachets.

The multi-microparticulate pharmaceutical formulation according to the invention makes it possible to ensure in a definite manner a delayed and sustained release of AP in the gastrointestinal tract, by virtue of two triggering factors and thus to escape the inter- and intra-individual variability in gastric emptying conditions, while at the same time being economically viable and easy to ingest (optimized compliance with the treatment).

Beyond the qualitative parameters defining the coated microparticles according to the invention, it may be pointed out that, in accordance with one advantageous quantitative embodiment, the inner coating film of these microparticles has the following quantitative weight percentage composition:

(A1) between 10 and 90 and preferably between 15 and 80,
(A2) between 5 and 50 and preferably between 10 and 40,
(A3) between 1 and 30 and preferably between 2 and 20,
(A4) between 0 and 20 and preferably between 0 and 15.

The coating film B represents not more than 50% and preferably not more than 40% by weight of the microparticles (or, in other words, the film B has a coating ratio TPB of less than or equal to 50% and preferably less than or equal to 40% by dry weight, relative to the total mass of coated microparticles).

According to one preferred characteristic of the invention, the two coating films A and B together represent not more than 50% by dry weight relative to the total mass of the coated microparticles.

These limited coating proportions make it possible to produce pharmaceutical formulation units each containing a high dose of active principle, without exceeding a prohibitive size with regard to swallowing. The compliance with the treatment and thus the success of the treatment cannot fail to be enhanced thereby.

The coated microparticles according to the invention comprise at least two coating films: an inner film directly in contact with the active principle particle, optionally one or more intermediate films, and an outer film, in contact with the inner film or, where appropriate, with an intermediate film.

According to one particular embodiment, the coating film A is an outer film and the coating film B is an inner film. According to another particular embodiment, the coating film A is an inner film, in contact with the active principle particle, and the coating film B is an outer film.

The choice of the appropriate structure depends especially on the type of active principle, the desired lag period or the rate of release. For example, for an acidic active principle requiring a long release time, a coating film B as inner film and a coating film A as outer film will be preferred.

Advantageously, the coated microparticles according to the invention comprise only two coating films: a coating film A and a coating film B. This makes it possible to achieve the objectives of the invention, in particular control of the release of the AP as a function of the pH and of time, these two mechanisms being independent of each other, while at the same time giving the coated microparticles a simple structure and maintaining small sizes.

As regards the structure of the coated microparticles according to the invention, two preferred embodiments of this structure are detailed hereinbelow, in a nonlimiting manner.

According to a first embodiment, at least some of the modified-release coated microparticles of active principle(s) each comprise:
a microparticle of active principle(s), coated with
at least one coating film A,
and at least one coating film B.

Preferably, the microparticle of active principle(s) is a granule comprising the active principle(s) and one or more pharmaceutically acceptable excipients.

According to a second embodiment, at least some of the modified-release coated microparticles of active principle(s) each comprise:
a neutral core,
at least one active layer comprising the active principle(s) and coating the neutral core,
at least one coating film A,
and at least one coating film B.

According to a first possibility, the neutral core may be, for example, a sugar-based (sucrose, dextrose, lactose or the like) neutral core, a cellulose microsphere or any other pharmaceutically acceptable particle with a mean diameter of less than 800 μm. Advantageously, the neutral core has a mean diameter of between 1 and 800 μm and preferably between 20 and 500 μm.

The active layer may optionally comprise, besides the active principle(s), one or more pharmaceutically acceptable excipients.

Advantageously, the standard pharmaceutically acceptable excipients known to those skilled in the art may especially be:
dyes;
plasticizers, for instance dibutyl sebacate;
hydrophilic compounds, for instance cellulose and derivatives thereof or polyvinylpyrrolidone and derivatives thereof;
and mixtures thereof.

As regards the preparation of the coated microparticles, the techniques advantageously used for depositing the coating allowing modified release of the active principle(s) or for depositing the active layer based on the active principle(s) are techniques known to those skilled in the art, for instance the technique of spray-coating in a fluidized-air bed, wet granulation, compacting and extrusion-spheronization.

The invention may be implemented independently of the solubility of the AP in water. Four classes of AP are defined, especially as a function of their solubility, according to the “Biopharmaceutics Classification System” (BCS) of the US Food and Drug Administration: Amido G. L. et al., “A theoretical basis for a biopharmaceutics drug classification: the correlation of in vivo drug product dissolution and in vivo bioavailability”, Pharmaceutical Research, vol. 12, pp. 413-420 (1995). APs belonging to these various classes may be used according to the present invention.

Qualitatively speaking, the AP contained in the coated microparticles according to the invention is absorbable essentially in the upper parts of the gastrointestinal tract and it is advantageously chosen from at least one of the following families of active substances: agents for treating alcohol abuse, agents for treating Alzheimer's disease, anesthetics, agents for treating acromegaly, analgesics, antiasthmatic agents, agents for treating allergies, anticancer agents, antiinflammatories, anticoagulants and antithrombotic agents, anticonvulsants, antiepileptic agents, anti-diabetic agents, antiemetic agents, antiglaucoma agents, antihistaminics, antiinfectious agents, antibiotics, antifungal agents, antiviral agents antiparkinson agents, anticholinergic agents, anti-tussive agents, carbonic anhydrase inhibitors, cardiovascular agents, hypolipemiants, antiarrhythmic agents, vasodilators, antiangina agents, antihypertensives, vasoprotective agents, cholinesterase inhibitors, agents for treating central nervous system disorders, central nervous system stimulants, contraceptives, fertility promoters, labor inducers and inhibitors, agents for treating mucoviscidosis, dopamine receptor agonists, agents for treating endometriosis, agents for treating erectile dysfunction, agents for treating fertility disorders, agents for treating gastrointestinal disorders, immunomodulators and immunosuppressants, agents for treating memory disorders, antimigraine agents, muscle relaxants, nucleoside analogs, agents for treating osteoporosis, parasympathomimetic agents, prostaglandins, psychotherapeutic agents, sedatives, hypnotics and tranquilizers, neuroleptic agents, anxiolytic agents, psychostimulants, antidepressants, dermatological treatment agents, steroids and hormones.

Examples of agents for treating acromegaly include: octreotide, laureotide and pegvisomant, and salts thereof, esters thereof, hydrates thereof, polymorphs thereof and isomers thereof.

Examples of agents for treating alcohol abuse include: chlorazepate, chlordiazepoxide, diazepam, disulfuram, hydroxyzine, naltrexone, and salts thereof, esters thereof, hydrates thereof, polymorphs thereof and isomers thereof.

Examples of anesthetics include: adrenalin, bupivacaine, chloroprocaine, desflurane, etidocaine, levobupivacaine, lidocaine, midazolam, propofol, ropivacaine, and salts thereof, esters thereof, hydrates thereof, polymorphs thereof and isomers thereof.

Examples of analgesics include: acetaminophen, aspirin, bupivacaine, buprenorphine, butorphanol, celecoxib, clofenadol, choline, clonidine, codeine, diflunisal, dihydrocodeine, dihydroergotamine, dihydromorphine, ethylmorphine, etodolac, eletriptan, eptazocine, ergotamine, fentanyl, fenoprofen, hyaluronic acid, hydrocodone, hydromorphone, hylane, ibuprofen, indomethacin, ketorolac, ketotifen, levomethadone, levallorphan, levorphanol, lidocaine, mefenamic acid, meloxicam, meperidine, methadone, morphine, nabumetone, nalbuphine, nefopam, nalorphine, naloxone, naltrexone, naproxen, naratriptan, nefazodone, mormethadone, oxapozine, oxycodone, oxymorphone, pentazocine, pethidine, phenpyramide, piritramide, piroxicam, propoxyphen, refecoxib, rizatriptan, ketoprofen, sulindac, sumatriptan, tebacone, tilidine, tolmetine, tramadol, zolmitriptan, and salts thereof, esters thereof, hydrates thereof, polymorphs thereof and isomers thereof.

Examples of antiasthmatic agents include: ablukast, azelastine, bunaprolast, cinalukast, cromitrile, cromolyne, enofelast, isambxole, ketotifen, levcromekaline, lodoxamide, montelukast, ontazolast, oxarbazole, oxatomide, piriprost potassium, pirolate, pobilukast, edamine, pranlukast, quazolast, repirinast, ritolukast, sulukast, tetrazolastmeglumine, tiaramide, tibenelast, tomelukast, tranilast, verlukast, verofylline, zarirlukast, and salts thereof, esters thereof, hydrates thereof, polymorphs thereof and isomers thereof.

Examples of anticancer agents include: adriamycin, aldesleukin, allopurinol, altretamine, amifostine, anastrozole, asparaginase, betamethasone, bexaroten, bicalutamide, bleomycin, busulfan, capecitabine, carboplatin, carmustine, chlorambucil, cisplatin, cladribine, conjugated estrogen, cortisone, cyclophosphamide, cytarabine, dacarbazine, daunorubicin, dactinomycin, denileukin, dexamethasone, discodermolide, docetaxel, doxorubicin, eloposidem, epirubicin, epoetin, epothilones, estramustine, esterified estrogen, ethynyl-estradiol, etoposide, exemestane, flavopirdol, fluconazole, fludarabine, fluorouracil, flutamide, floxuridine, gemcitabine, gemtuzumab, goserelin, hexamethylmelamine, hydrocortisone, hydroxyurea, idarubicin, ifosfamide, interferon, irinotecan, lemiposide, letrozole, leuprolide, levamisole, levothyroxine, lomustine, mechlorethamine, melphalan, mercaptopurine, megestrol, methotrexate, methylprednisolone, methyltestosterone, mithramycin, mitomycin, mitotane, mitoxantrone, mitozolomide, mutamycin, nilutamide, paclitaxel, pamidronate, pegaspargase, pentostatin, plicamycin, porfimer, prednisolone, procarbazine, rituximab, sargramostim, semustine, streptozocin, tamoxifen, temozolamide, teniposide, testolactone, thioguanine, thiotepa, tomudex, topotecan, toremifen, trastumuzab, tretinoin, semustine, streptozolocin, valrubicin, verteprofin, vinblastine, vincristine, vindesine, vinorelbine, and salts thereof, esters thereof, hydrates thereof, polymorphs thereof and isomers thereof.

Examples of anticoagulants and antithrombotic agents include: warfarin, dalteparine, heparine, tinzaparin, enoxaparin, danaparoid, abciximab, alprostadil, altiplase, anagralide, anistreplase, argatroban, ataprost, betaprost, camonagrel, cilostazol, clinprost, clopidogrel, cloricromen, dermatan, desirudine, domitroban, drotaverine, epoprostenol, eptifibatide, fradafiban, gabexate, iloprost, isbogrel, lamifiban, lamoteplase, lefradafiban, lepirudin, levosimendan, lexipafant, melagatran, nafagrel, nafamostsat, nizofenone, orbifiban, ozagrel, pamicogrel, parnaparin, quinobendan, reteplase, sarpogralate, satigrel, silteplase, simendan, ticlopidine, vapiprost, tirofiban, xemilofiban, Y20811, and salts thereof, esters thereof, hydrates thereof, polymorphs thereof and isomers thereof.

Examples of anticonvulsants include: carbamazepine, clonazepam, clorazepine, diazepam, divalproex, ethosuximide, ethotion, felbamate, fosphenyloin, gabapentine, lamotrigine, levetiracetam, lorazepam, mephenyloin, mephobarbital, metharbital, methsuximide, oxcarbazepine, phenobarbital, phenyloin, primidone, tiagabine, topiramate, valproic acid, vigabatrin, zonisamide, and salts thereof, esters thereof, hydrates thereof, polymorphs thereof and isomers thereof.

Examples of antidiabetic agents include: acarbose, acetohexamide, carbutamide, chlorpropamide, epalrestat, glibornuride, gliclazide, glimepiride, glipizide, gliquidone, glisoxepide, glyburide, glyhexamide, metformin, miglitol, nateglinide, orlistat, phenbutamide, pioglitazone, repaglinide, rosiglitazone, tolazamide, tolbutamide, tolcyclamide, tolrestat, troglitazone, voglibose, and salts thereof, esters thereof, hydrates thereof, polymorphs thereof and isomers thereof.

Examples of antiemetic agents include: alprazolam, benzquinamide, benztropine, betahistine, chlorpromazine, dexamethasone, difenidol, dimenhydrinate, diphenhydramine, dolasetron, domperidone, dronabinol, droperidol, granisetron, haloperidol, lorazepam, meclizine, methylprednisolone, metoclopramide, ondansetron, perphenazine, prochlorperazine, promethazine, scopolamine, tributin, triethylperazine, triflupromazine, trimethobenzamide, tropisetron, and salts thereof, esters thereof, hydrates thereof, polymorphs thereof and isomers thereof.

Examples of antiglaucoma agents include: alprenoxime, dapiprazole, dipivefrin, latanoprost, naboctate, pimabine, and salts thereof, esters thereof, hydrates thereof, polymorphs thereof and isomers thereof.

Examples of antihistaminics include: acepromazine, acrivastine, activastine, albuterol, alimemazine, antazoline, azelastine, bitolterol, amlexanox, benzydamine, brompheniramine, cetirizine, chlorpheniramine, cimetidine, cinnarizine, clemastine, clofedanol, cycloheptazine, cyproheptadine, diclofenac, difencloxazine, diphenhydramine, dotarizine, ephedrine, epinastine, epinephrine, ethylnorepinephrine, etybenzatropine, fenpentadiol, fenpoterol, fexofenadine, flurbiprofen, hydroxyzine, isoetharine, isoproterenol, ipratropium bromide, ketorolac, levocetirizine, levomepromazine, loratidine, mequitazine, metaproterenol, niaprazine, oxatomide, oxomemazine, phenylephrine, phenylpropanolamine, pirbuterol, promethazine, pseudoephedrine, pyrilamine, salmeterol, terbutaline, terfenadine, tranilast, xanthine derivatives, xylometazoline, and salts thereof, esters thereof, hydrates thereof, polymorphs thereof and isomers thereof.

Examples of antiinfectious agents, especially antibiotics, antifungal agents and antiviral agents, include: abacavir, aciclovir, albendazole, amantadine, amphotericin, amikacin, aminosalicylic acid, amoxycillin, ampicillin, amprenavir, atovaquine, azithromycin, aztreonam, carbenicillin, cefaclor, cefadroxil, cefamandole, cefazolin, cefdinir, cefepime, cefexime, cefoperazone, cefotaxime, cefotitam, cefoperazone, cefoxitine, cefpodoxine, cefprozil, ceftazidime, ceftibuten, ceftizoxime, ceftriaxone, cefuroxime, cephalexine, chloroquine, cidofovir, cilastatin, ciprofloxacin, clarithromycin, clavulanic acid, clindamycin, colistimethate, dalfopristin, dapsone, daunorubicin, delavirdine, demeclocycline, didanosine, doxycycline, doxorubicin, efavirenz, enoxacin, erythromycin, ethambutol, ethionamide, famcyclovir, fluconazole, flucytocine, foscarnet, fosfomycin, ganciclovir, gatifloxacin, griseofulvin, hydroxychloroquine, imipenem, indinavir, interferon, isoniazide, itraconazole, ivermectil, ketoconazole, lamivudin, levofloxacin, linizolide, lomefloxacin, loracarbef, mebendazole, mefloquine, meropenem, methanamine, metronidazole, minocycline, moxefloxacin, naldixic acid, nelfinavir, neomycin, nevirapine, nitorfurantoin, norfloxacin, ofloxacin, oseltamivir, oxytetracycline, palivizumab, penicillin, perfloxacin, piperacillin, praziquantel, pyrazinamide, pyrimethamine, quinidine, quinupristine, retonavir, ribavirin, rifabutin, rifampicin, rimantadine, saquinavir, sparfloxacin, stavudin, streptomycin, sulfamethoxazole, tetramycin, terbinafine, tetracycline, ticarcillin, thiabendazole, tobramycin, trimethoprim, trimetraxate, troleandomycin, trovafloxacin, valaciclovir, vancomycin, zalcitabine, zanamivir, zidovudine, and salts thereof, esters thereof, hydrates thereof, polymorphs thereof and isomers thereof.

Examples of antiparkinson agents include: amantadine, adrogolide, altinicline, benzatropine, biperiden, brasofensine, bromocriptine, budipine, cabergoline, CHF-1301, dihydrexidine, entacapone, etilevodopa, idazoxane, iometopane, lazabemide, melevodopa, carbidopa, levodopa, mofegiline, moxiraprine, pergolide, pramipexole, quinelorane, rasagiline, ropinirole, seligiline, talipexole, tolcapone, trihexyphenidyl, and salts thereof, esters thereof, hydrates thereof, polymorphs thereof and isomers thereof.

Examples of antirheumatic agents include: azathiprine, betamethasone, celecoxib, cyclosporine, diclofenac, hydroxychloroquine, indomethacin, infliximab, mercaptobutanedioic acid, methylprednisolone, naproxen, penicillamine, piroxicam, prednisolone, sulfasalazine, and salts thereof, esters thereof, hydrates thereof, polymorphs thereof and isomers thereof.

Examples of anti-platelet-aggregating agents include: abciximab, anagrelide, aspirin, cilostazol, clopidogrel, dipyridamole, epoprostenol, eptifibatide, ticlopidine, tinofiban, and salts thereof, esters thereof, hydrates thereof, polymorphs thereof and isomers thereof.

Examples of antispasmodic and anticholinergic agents include: aspirin, atropine, diclofenac, hyoscyamine, mesoprostol, methocarbamol, phenobarbital, scopolamine, and salts thereof, esters thereof, hydrates thereof, polymorphs thereof and isomers thereof.

Examples of antitussive agents include: acetaminophen, acrivastine, albuterol, benzonatate, beractant, brompheniramine, caffeine, calfactant, carbetapentane, chlorpheniramine, codeine, colfuscerine, dextromethorpham, dornase alpha, doxylamine, epinephrine, fexofenadine, guaphenesin, ipratropium, levalbuterol, metaproterenol, montelukast, pentoxyphilline, phenylephrine, phenylpropanolamine, pirbuterol, poractant alpha, pseudoephedrine, pyrilamine, salbuterol, salmeterol, terbutaline, theophylline, zafirlukast, zileuton, and salts thereof, esters thereof, hydrates thereof, polymorphs thereof and isomers thereof.

Examples of carbonic anhydrase inhibitors include: acetazolamide, dichlorphenamide, dorzolamide, methazolamide, sezolamide, and salts thereof, esters thereof, hydrates thereof, polymorphs thereof and isomers thereof.

Examples of cardiovascular agents, especially hypolipemiants, antiarrhythmic agents, vasodilators, antiangina agents, antihypertensives and vasoprotective agents, include: abciximab, acebutolol, activase, adenosine, adrenaline, amidarone, amiloride, amlodipine, amyl nitrate, atenolol, atorvastatin, benzepril, bepiridil, betaxalol, bisoprolol, candesartan, captopril, cartenolol, carvedilol, cerivastatin, chlorthalidone, chlorthiazole, clofibrate, clonidine, colestipol, colosevelam, digoxin, diltiazem, disopyramide, dobutamine, dofetilide, doxazosine, enalapril, epoprostenol, eprosartan, esmolol, ethacrynate, erythrityl, felodipine, fenoidapam, fosinopril, flecamide, fluorosemide, fluvastatin, gemfibrozil, hydrochlorthiazide, hydroflumethazine, ibutilide, indapamide, isosorbide, irbesartan, labetolol, lacidipine, lisinopril, losartan, lovastatin, mecamylamine, metoprolol, metaminol, metazolone, methylchlothiazide, methyldopa, metyrosine, mexiletine, midrodine, milrinone, moexipril, nadolol, niacin, nicardipine, nicorandil, nifedipine, nimodipine, nisoldipine, nitroglycerine, phenoxybenzamine, perindopril, polythiazide, pravastatin, prazosin, procainamide, propafenone, propranolol, quanfacine, quinapril, quinidine, ranipril, reteplase, simvastatin, sotalol, spironolactone, streptokinase, telmisartan, terazosin, timolol, tocainamide, torsemide, trandolapril, triamteren, trapidil, valsartan, and salts thereof, esters thereof, hydrates thereof, polymorphs thereof and isomers thereof.

Examples of vasodilators include: adenosine, alverine, caffeine, dihydroergocornine, enalapril, enoximone, iloprost, kalleone, lidoflazine, nicardipine, nimodipine, nicotinic acid, papaverine, pilocarpine, salbutamol, theophylline, trandolapril, uradipil, vincamine, and salts thereof, esters thereof, hydrates thereof, polymorphs thereof and isomers thereof.

Examples of cholinesterase inhibitors include: donepezil, edrophonium, neostigmine, pyridostigmine, rivastigmine, tacrine, and salts thereof, esters thereof, hydrates thereof, polymorphs thereof and isomers thereof.

Examples of central nervous system stimulants include: caffeine, doxapram, dexoamphetamine, donepezil, edorphonium, methamphetamine, methylphenidate, modafinil, neostigmine, pemoline, phentermine, pyridostigmine, rivastigmine, tacrine, and salts thereof, esters thereof, hydrates thereof, polymorphs thereof and isomers thereof.

Examples of contraceptives include: desogestral, ethinyl-estradiol, ethynodiol, levonorgestrel, medroxyprogesterone, mestranol, norgestimate, norethindrone, norgestrel, and salts thereof, esters thereof, hydrates thereof, polymorphs thereof and isomers thereof.

Examples of mucoviscidosis treatment agents include: domase alpha, pancrelipase, tobramycin, and salts thereof, esters thereof, hydrates thereof, polymorphs thereof and isomers thereof.

Examples of dopamine receptor agonists include: amantadine, cabergoline, fenoldopam, pergolide, pramipezal, ropinirole, and salts thereof, esters thereof, hydrates thereof, polymorphs thereof and isomers thereof.

Examples of endometriosis treatment agents include: danazol, goserelin, leuprolide, nafarelin, norethindrone, and salts thereof, esters thereof, hydrates thereof, polymorphs thereof and isomers thereof.

Examples of erectile dysfunction treatment agents include alprostadil, sildenafil, yohimbine, and salts thereof, esters thereof, hydrates thereof, polymorphs thereof and isomers thereof.

Examples of fertility treatment agents include: citrorelix, clomiphen, follitropin, ganirelix, gonadotropin, menotropin, progesterone, urofollitropin, and salts thereof, esters thereof, hydrates thereof, polymorphs thereof and isomers thereof.

Examples of gastrointestinal disorder treatment agents include: alosetron, bisacodyl, bismuth subsalicylate, celecoxib, cimetidine, difoxine, dipheoxylate, docusate, esomeprazole, famotidine, glycopyrrolate, infliximab, lansoprazole, loperamide, metaclopramide, nizatidine, omeprazole, pantoprazole, rabeprazole, ranitidine, simethicone, sucralfate, and salts thereof, esters thereof, hydrates thereof, polymorphs thereof and isomers thereof.

Examples of immunomodulators and immunosuppressants include: azathioprine, ceftizoxine, cyclosporine, daclizumab, glatiramer, immunoglobulin, interferon, leflunomide, levamisol, mycophenolate, phthalidomide, ribavirine, sirolimus, and salts thereof, esters thereof, hydrates thereof, polymorphs thereof and isomers thereof.

Examples of Alzheimer's disease treatment agents include: CP 118954, donepezil, galanthamine, metrifonate, revastigmine, tacrine, TAK-147, and salts thereof, esters thereof, hydrates thereof, polymorphs thereof and isomers thereof.

Examples of antimigraine agents include: acetaminophen, dihydroergotamine, divalproex, ergotamine, propranolol, risatriptan, sumatriptan, trimetrexate, and salts thereof, esters thereof, hydrates thereof, polymorphs thereof and isomers thereof.

Examples of muscle relaxants include: alcuronium chloride, azapropazone, atracurium, baclofen, carisoprodol, quinine derivatives, chloromezanone, chlorophenesincarbamate, chlorozoxazone, cyclobenzaprine, dantrolen, decamethonium bromide, dimethyltubocurarinium chloride, doxacurium, fenyramidol, gallamine triethiodide, guaiphenesin, hexafluorenium bromide, hexacarbacholine bromide, memantin, mephenesin, meprobamate, metamisol, metaxalone, methocarbamol, mivacurium, orphenadrine, pancuronium, phenazone, phenprobamate, pipecuronium, rapacuronium, rocuronium, succinylcholine, suxamethonium chloride, tetrazepam, tizanidine, tubocurarine chloride, tybamate, vecuronium, and salts thereof, esters thereof, hydrates thereof, polymorphs thereof and isomers thereof.

Examples of nucleoside analogs include: abacavir, aciclovir, didanosine, gamciclovir, gemcitabine, lamivudine, ribavirin, stavudine, zalcitabine, and salts thereof, esters thereof, hydrates thereof, polymorphs thereof and isomers thereof.

Examples of osteoporosis treatment agents include: alendronate, calcitonin, estradiol, estropipate, medroxyprogesterone, norethindrone, norgestimate, pamidronate, raloxifen, risdronate, zoledronate, and salts thereof, esters thereof, hydrates thereof, polymorphs thereof and isomers thereof.

Examples of parasympathomimetic agents include: bethanechol, piperidine, edrophonium, glycopyrolate, hyoscyamine, pilocarpine, tacrine, yohimbine, and salts thereof, esters thereof, hydrates thereof, polymorphs thereof and isomers thereof.

Examples of prostaglandins include: alprostadil, epoprostenol, misoprostol, and salts thereof, esters thereof, hydrates thereof, polymorphs thereof and isomers thereof.

Examples of psychotherapeutic agents include: acetophenazine, alentemol, alpertine, alprazolam, amitriptyline, apriprazole, azaperone, batelapine, befipiride, benperidol, benzindopyrine, bimithil, biriperone, brofoxine, bromperidol, broniperidol, bupropione, buspirone, butaclamol, butaperazine, butaperazin, carphenazine, carvotroline, cericlamine, chlorazepine, chlordiazepoxide, chlorpromazine, chlorprothixen, cinperen, cintriamide, citalopram, clomacran, clonazepam, clopenthixol, clopimozide, clopipazan, cloroperone, clothiapine, clothixamide, clozapine, cyclophenazine, dapiprazole, dapoxetine, desipramine, divalproex, dipyridamole, doxepin, droperidol, duloxetine, eltoprazine, eptipirone, etazolate, fenimide, flibanserine, flucindole, flumezapine, fluoxetine, fluphenazine, fluspiperone, fluspirilen, flutroline, fluvoxamine, gepirone, gevotroline, halopemide, haloperidol, hydroxyzine, hydroxynortriptyline, iloperidone, imidoline, lamotrigine, loxapine, enperone, mazapertine, mephobarbital, meprobamate, mesoridazine, mesoridazine, milnacipran, mirtazepine, metiapine, milenperone, milipertine, molindone, nafadotride, naranol, nefazodone, neflumozide, ocaperidone, odapipam, olanzapine, oxethiazine, oxiperomide, pagoclone, paliperidone, paroxiten, penfluridol, pentiapine, perphenazine, phenelzine, pimozide, pinoxepin, pipamperone, piperacetazine, pipotiazine, piquindone, pirlindole, pivagabine, pramipexole, prochlorperazine, promazine, quetiapine, reboxetine, remoxipride, risperidone, rimcazole, robolzotan, selegiline, seperidol, sertraline, sertindole, seteptiline, setoperone, spiperone, sunipitrone, tepirindole, thioridazine, thiothixen, tiapride, tioperidone, tiospirone, topiramate, tranylcypromine, trifluoperazine, trifluperidol, triflupromazine, trimipramine, venlafaxine, ziprasidone, and salts thereof, esters thereof, hydrates thereof, polymorphs thereof and isomers thereof.

Examples of sedatives, hypnotics and tranquillizers include: bromazepam, buspirone, clazolam, clobazam, chlorazepate, diazepam, demoxepam, dexmedetomidine, diphenyhydramine, doxylamine, enciprazine, estrazolam, hydroxyzine, ketazolam, lorazatone, lorazepam, loxapine, medazepam, meperidine, methobarbital, midazolam, nabilone, nisobamate, oxazepam, pentobarbital, promethazine, propofol, triazolam, zaleplon, zolpidem, and salts thereof, esters thereof, hydrates thereof, polymorphs thereof and isomers thereof.

Examples of dermatological treatment agents include: acitretin, alclometasone, alitretinoin, betamethasone, calciprotrine, chlorhexidine, clobetasol, clocortolone, clotriamozole, collagenase, cyclosporine, desonide, difluorosone, doxepine, eflomithine, finasteride, fluocinolone, flurandrenolide, fluticasone, halobetasol, hydrochloroquine, hydroquinone, hydroxyzine, ketoconazole, mafenide, malathion, menobenzone, neostigmine, nystatin, podofilox, povidone, tazoroten, tretinoin, and salts thereof, esters thereof, hydrates thereof, polymorphs thereof and isomers thereof.

Examples of steroids and hormones include: alclometasone, betamethasone, calcitonine, citrorelix, clobetasol, clocortolone, cortisones, danazol, desmopressin, desonide, desogestrel, desoximetasone, dexamethasone, diflorasone, estradiol, estrogens, estropipate, ethynilestradiol, fluocinolone, flurandrenolide, fluticasone, glucagon, gonadotropin, goserelin, halobetasol, hydrocortisone, leuprolide, levonorgestrel, levothyroxine, medroxyprogesterone, menotropins, methylprednisolone, methyltestosterone, mometasone, naferelin, norditropin, norethindrone, norgestrel, octreolide, oxandrolone, oxymetholone, polytropin, prednicarbate, prednisolone, progesterone, sermorelin, somatropin, stanozolol, testosterone, urofollitropin, and salts thereof, esters thereof, hydrates thereof, polymorphs thereof and isomers thereof.

Reference may also be made to the list of active principles given in patent application EP-A-0 609 961 on pages 4 to 8.

According to another of its aspects, the invention relates to a medicament or a pharmaceutical, veterinary or dietetic formulation, characterized in that it comprises a plurality of coated microparticles as defined above, for example at least 500, preferably from 1000 to 1 000 000 and even more preferentially from 5000 to 500 000 microparticles.

The medicament according to the invention is thus multi-microparticulate, i.e. it comprises at least microparticles constituted of microparticles of coated active principle(s). These microparticles of active principle(s) may be, for example, crude (pure) active principle(s) in pulverulent form, matrix granules of active principle(s) with various other ingredients or neutral microspheres covered with at least one layer comprising active principle, as is explained hereinabove.

The modified-release coated AP microparticles may be likened to microunits containing at least one active principle and forming at least part of the constituent elements of the medicament according to the invention.

These microparticles are all the more advantageous since they are also fully tolerated by the body, especially at the gastric level, and may moreover be obtained easily and economically.

Each coated microparticle may comprise one or more active principles.

The medicament or formulation according to the invention may comprise at least one active principle in an immediate-release form, for example microunits of active principle other than coated microparticles. They may be, for example, microparticles with immediate release of active principle(s). These microparticles may be, for example, uncoated microparticles of active principle(s) of the same type as the microparticles that are useful in the preparation of the coated microparticles according to the invention.

The active principle in an immediate-release form may be identical to or different from the active principle(s) contained in the coated microparticles. Each immediate-release microparticle may comprise one or more active principles.

Thus, according to one variant of the invention, the medicament or the formulation comprises at least one active principle in an immediate-release form that is the same as an active principle contained in at least some of the coated microparticles.

In addition, all the microunits (microparticles and/or coated microparticles) constituting the medicament according to the invention may be formed by different populations of microunits, these populations differing from each other at least by the nature of the active principle(s) contained in these microunits and/or by the amount of optional active principle(s) they contain and/or by the composition of the coating films A and/or B and/or by the fact that they display modified release or immediate release.

The invention is thus directed toward a medicament or a pharmaceutical, veterinary or dietetic formulation comprising a plurality of populations of microparticles (coated or uncoated), said populations differing from each other by their lag time and/or by their triggering pH and/or by their rate of release and/or by the active principle they contain.

Without wishing to be limiting, it should nevertheless be pointed out that the medicament according to the invention is particularly advantageous in that it may be:
in the form of a single daily oral dose comprising from 500 to 500 000 microunits, some of which contain active principle(s);
in the form of a single daily oral dose comprising from 500 to 500 000 microparticles (coated and/or uncoated) with modified release of the active principle(s).

Advantageously, the pharmaceutical, veterinary or dietetic formulation comprising coated microparticles according to the invention is in a pharmaceutical form chosen from the group comprising: tablets (advantageously orodispersible or gastrodispersible tablets), powders, suspensions, syrups, powders for suspension to be reconstituted, or gel capsules.

It may be advantageous to mix together in the same gel capsule, in the same tablet or in the same powder at least two types of coated microparticle whose release kinetics are different but which are included in the characteristic context of the invention.

The coated microparticles according to the invention may also be mixed with a certain amount of AP that is immediately available in the body. It may also be envisioned to combine coated microparticles containing different APs.

The invention also relates to the use of the coated microparticles described above for the manufacture of novel medicaments or pharmaceutical, veterinary or dietetic preparations of various APs, having optimized therapeutic or dietetic performance qualities and preferably being in the form of tablets, advantageously orodispersible or gastrodispersible tablets, powders or gel capsules.

The present invention also relates to these novel pharmaceutical, veterinary or dietetic preparations per se, which are novel in their structure, their presentation and their composition. Such pharmaceutical, veterinary or dietetic preparations are administered orally, preferably in single daily doses.

Finally, the invention is also directed toward a therapeutic treatment process, characterized in that it consists in ingesting, at a determined dosage, a medicament comprising the coated microparticles as defined above.

The invention will be explained more clearly by the examples hereinbelow, which are given solely for illustrative purposes to allow the invention to be clearly understood and to highlight its embodiment and/or implementation variants, and also its various advantages.

### BRIEF DESCRIPTION OF THE FIGURES

FIG. 1 shows the in vitro release profiles of the coated microparticles of Example 2. The profiles are curves of the weight percentage (% dissolved) of metformin HCl dissolved as a function of the time T in hours.

at pH=7.1: [symbol] at pH=1.4: [symbol] at variable pH: ---O---

FIG. 2 shows the in vitro release profiles of the coated microparticles of Example 3. The profiles are curves of the weight percentage (% dissolved) of metformin HCl dissolved as a function of the time T in hours.

at pH=7.1: [symbol] at pH=1.4: [symbol] at variable pH: ---O---

FIG. 3 shows the in vitro release profiles of the coated microparticles of Example 4. The profiles are curves of the weight percentage (% dissolved) of metformin HCl dissolved as a function of the time T in hours.

at pH=7.1: [symbol] at pH=1.4: [symbol] at variable pH: ---O---

FIG. 4 shows the in vitro release profiles at variable pH, of Examples 2, 3 and 4. The profiles are curves of the weight percentage (% dissolved) of metformin HCl dissolved as a function of the time T in hours.

at variable pH: Example 2: [symbol] Example 3: [symbol] Example 4: ---O---

FIG. 5 shows the in vitro release profiles of the coated microparticles of Example 5. The profiles are curves of the weight percentage (% dissolved) of metformin HCl dissolved as a function of the time T in hours.

at pH=7.1: [symbol] at pH=1.4:

[symbol]

FIG. 6 shows the in vitro release profiles of the coated microparticles of Example 6. The profiles are curves of the weight percentage (% dissolved) of aciclovir dissolved as a function of the time T in hours.

at pH=7.1: [symbol] at pH=1.4:

[symbol]

### EXAMPLES

Examples 1 to 5 use metformin HCl, which is an AP that is highly water-soluble. Example 6 uses aciclovir, which is an AP that is sparingly water-soluble.

Example 2 is a comparative example of preparation of coated microparticles comprising only one coating layer B. In Examples 3, 4 and 6, coated microparticles that comprise an inner coating layer A and an outer coating layer B are prepared. In Example 5, coated microparticles that comprise an inner coating layer B and an outer coating layer A are prepared.

In the examples that follow, the commercial names of the excipients mentioned find their chemical correspondence in the following table:

| Commercial name | Chemical name/monograph |
|---|---|
| Cremophor RH 40 | Macrogolglyceroli hydroxystearas |
| Klucel EF | Hydroxypropyl cellulose |
| Plasdone K29/32 | Povidone |
| EUDRAGIT L100-55 | Poly(methacrylic acid, ethyl acrylate) 1:1 |
| Kollicoat MAE 100P | Poly(methacrylic acid, ethyl acrylate) 1:1 |
| Acrycoat L100D | Poly(methacrylic acid, ethyl acrylate) 1:1 |
| EUDRAGIT S100 | Poly(methacrylic acid, methyl methacrylate) 1:2 |
| Ethocel 20P | Ethylcellulose |

In the examples, the dissolution tests are performed in a paddle dissolutest in accordance with European Pharmacopeia 5.2 or apparatus of type II in US Pharmacopeia 28-NF 23, maintained at 37° C. and rotated at 100 rpm.

### Example 1

### Preparation of Metformin, HCl Granules

1795.5 g of metformin HCl (Chemsource) and 94.5 g of povidone are dissolved in 2610 g of water. The solution is sprayed onto 210 g of neutral microspheres (NP Pharm) in a Glatt® GPCG3 spray coater.

### Example 2

### Example According to the Prior Art of Coated Microparticles Leading to a Delayed Release of Metformin, HCl (Comparative Example)

78.0 g of hydrogenated plant oil Type 1 NF (JRS Pharma) and 117.0 g of EUDRAGIT® L100-55 (Röhm) are hot-dissolved in 1756.0 g of ethanol. 1140 g of this solution are sprayed onto 455.0 g of metformin HCl microgranules prepared in Example 1 in a Glatt® GPCG3 spray coater. Coated microparticles are obtained.

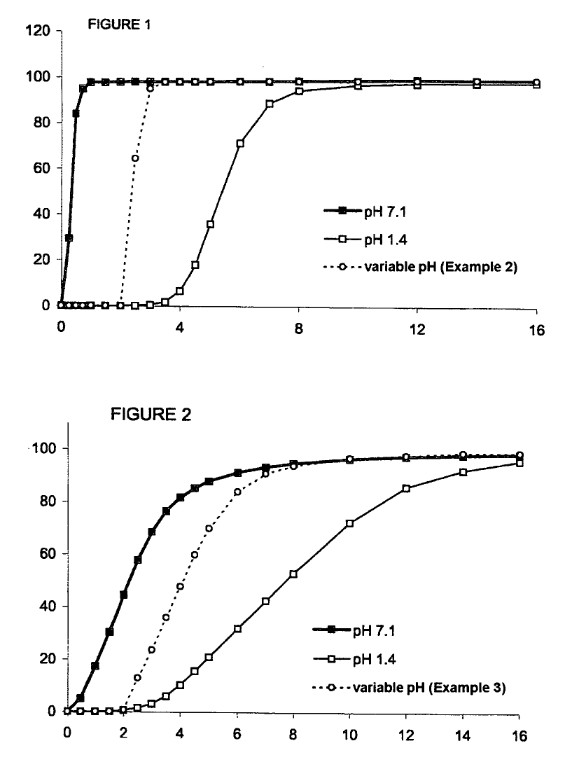

Dissolution tests were performed on the coated microparticles in the following media: i) HCl solution at pH 1.4, ii) KH₂PO₄/NaOH buffered solution at pH 7.1, and iii) HCl solution at pH 1.4 for 2 hours and then in KH₂PO₄/NaOH buffer medium at pH 7.1. The results of the dissolution tests are represented in FIG. 1.

### Example 3

### Preparation According to the Invention of Coated Microparticles Leading to a Delayed and Sustained Release of Metformin, HCl

517 g of microgranules of Example 1 are film-coated with 632 g of the following solution: 24.4 g of ethylcellulose, 2.6 g of povidone, 3.3 g of magnesium stearate, 2.6 g of castor oil, 627 g of ethanol.

45.5 g of hydrogenated plant oil Type 1 NF (JRS Pharma) and 68.3 g of EUDRAGIT® L100-55 (Röhm) are hot-dissolved in 1024 g of ethanol. 650 g of this solution are then sprayed onto 455 g of the microparticles obtained above. The film coating is performed in a Glatt® spray coater. Coated microparticles are obtained.

Dissolution tests were performed on the coated microparticles in the following media: i) HCl solution at pH 1.4, ii) KH₂PO₄/NaOH buffered solution at pH 7.1, and iii) HCl solution at pH 1.4 for 2 hours and then KH₂PO₄/NaOH buffer medium at pH 7.1. The results of the dissolution tests are shown in FIG. 2.

### Example 4

### Preparation According to the Invention of Coated Microparticles Leading to a Delayed and Sustained Release of Metformin, HCl

500 g of the microgranules of Example 1 are film-coated with 995 g of the following solution: 60.8 g of ethylcellulose, 3.2 g of povidone, 8.0 g of magnesium stearate, 8.0 g of castor oil, 925 g of ethanol.

45.5 g of hydrogenated plant oil Type 1 NF (JRS Pharma), 22.8 g of EUDRAGIT® L100-55 (Röhm) and 45.5 g of EUDRAGIT® S100 (Röhm) are hot-dissolved in 1024 g of ethanol. 650 g of this solution are then sprayed onto 455 g of the microparticles obtained above. The film coating is performed in a Glatt® spray coater. Coated microparticles are obtained.

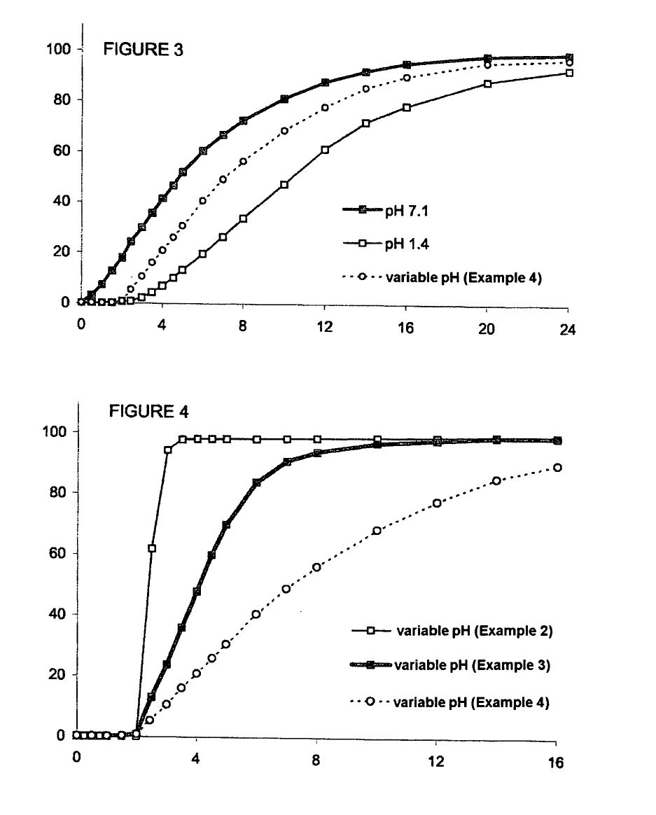

Dissolution tests were performed on the coated microparticles in the following media: i) HCl solution at pH 1.4, ii) KH₂PO₄/NaOH buffered solution at pH 7.1, and iii) HCl solution at pH 1.4 for 2 hours and then KH₂PO₄/NaOH buffer medium at pH 7.1. The results of the dissolution tests are shown in FIG. 3.

### Example 5

### Preparation According to the Invention of Coated Microparticles Leading to a Delayed and Sustained Release of Metformin, HCl

60.0 g of hydrogenated plant oil Type 1 NF (Abitec) and 90.0 g of Acrycoat L100D (NP Pharm) are hot-dissolved in 1350 g of isopropanol. The solution is sprayed onto 850 g of the metformin microgranules prepared in Example 1, in a Glatt® GPCG1 spray coater. 455 g of the microparticles obtained are then film-coated with 632 g of the following solution: 117 g of ethylcellulose, 66.3 g of povidone, 11.7 g of castor oil, 2242.5 g of isopropanol. Coated microparticles are obtained.

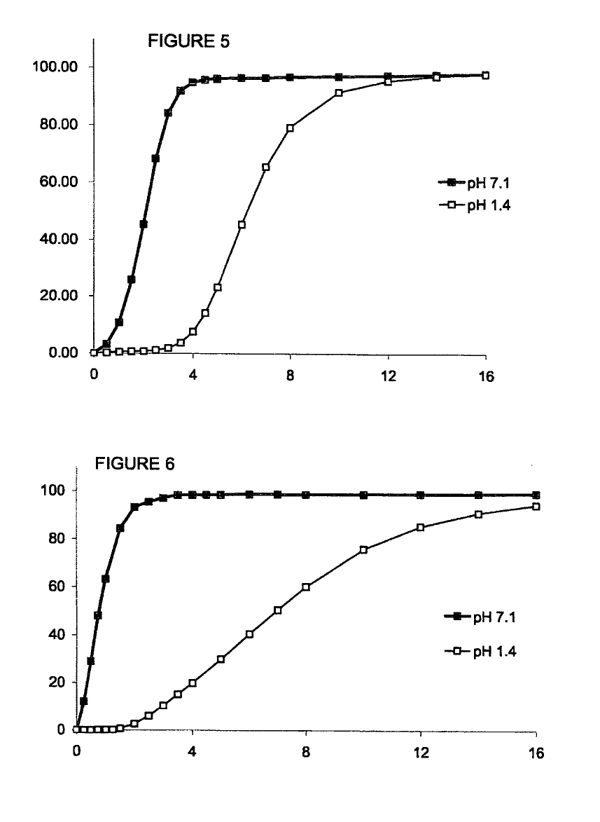

Dissolution tests were performed on the coated microparticles in the following media: i) HCl solution at pH 1.4, ii) KH₂PO₄/NaOH buffered solution at pH 7.1. The results of the dissolution tests are shown in FIG. 5.

### Example 6

### Preparation According to the Invention of Coated Microparticles Leading to a Delayed and Sustained Release of Aciclovir

320.0 g of aciclovir and 80.0 g of PVP are dissolved in a mixture containing 668.6 g of ethanol and 74.3 g of water. The solution is sprayed onto 1600 g of neutral microspheres (NP Pharm) in a Glatt® GPCG1.1 spray coater.

500 g of these microgranules are film-coated with the following solution: 21.1 g of ethylcellulose, 8.3 g of PVP, 2.5 g of castor oil, 606.4 g of ethanol.

45.5 g of hydrogenated plant oil Type 1 NF (Condea) and 68.3 g of Kollicoat MAE 100P (BASF) are hot-dissolved in 1023 g of ethanol. 867 g of this solution are then sprayed onto 455 g of the microparticles obtained above in a Glatt® GPCG1.1 spray coater. Coated microparticles are obtained.

Dissolution tests were performed on the coated microparticles in the following media: i) HCl solution at pH 1.4, ii) KH₂PO₄/NaOH buffered solution at pH 7.1. The results of the dissolution tests are shown in FIG. 6.

As shown in FIG. 4, in the microparticles according to the prior art, the release of the AP was immediate as soon as the release-triggering pH was reached. On the other hand, with the coated microparticles in accordance with the invention, when the release-triggering pH is reached, the release of the AP is sustained and controlled.

As shown in FIGS. 2 to 5 and 6, the coated microparticles according to the invention effectively make it possible to sustain and control the release of a highly soluble AP (metformin, HCl, FIGS. 2 to 5) and of a sparingly soluble AP (aciclovir, FIG. 6).

1. Coated “reservoir” microparticles containing at least one active principle (AP) and being of the type:
constituted by AP particles each covered with at least two different coating films,
with a mean diameter of less than 2000 microns, preferably between 50 and 800 microns and even more preferentially between 100 and 600 microns;
characterized in that, in combination, the coating films are capable:
of ensuring release of the AP governed by two different triggering mechanisms, one based on a variation in pH and the other allowing the release of the AP after a predetermined residence time in the stomach,
of inducing in vitro dissolution behavior (performed in a paddle dissolutest in accordance with the European Pharmacopeia 5.2 or a device of type II in US Pharmacopeia 28-NF23, maintained at 37° C. and rotated at 100 rpm) such that:
at constant pH 1.4, the dissolution profile comprises a lag phase of adjustable duration of less than or equal to 8 hours, preferably less than or equal to 5 hours and even more preferentially between 1 and 5 hours;
the passage from pH 1.4 to pH 7.1 leads to a sustained-release phase of adjustable duration, starting without a lag time and such that t½ is between 0.25 and 20 hours, preferably between 0.25 and 12 hours, more preferably between 0.25 and 8 hours and even more preferentially between 0.25 and 4 hours, in which t½ is the time required to release 50% of at least one of the active principles contained in the coated microparticles.

2. The microparticles as claimed in claim 1, characterized in that each of them comprises:
at least one coating film (A) having the following composition:
(A1) at least one film-forming (co)polymer (A1) that is insoluble in the liquids of the gastrointestinal tract;
(A2) at least one (co)polymer (A2) that is soluble in the liquids of the gastrointestinal tract;
(A3) at least one plasticizer (A3);
(A4) optionally at least one surfactant and/or lubricant (A4);
and at least one coating film (B) constituted of a composite material comprising at least one hydrophilic polymer (B1) bearing groups that are ionized at neutral pH and at least one hydrophobic compound (B2).

3. The microparticles as claimed in claim 2, characterized in that each of them comprises a coating film (A) and a coating film (B).

4. The microparticles as claimed in claim 2 or 3, in which the coating film A has a coating ratio (Tp_{A})—expressed as a dry weight % relative to the total mass of the microparticles—such that Tp_{A}≧2%, preferably Tp_{A}≧3% and even more preferentially Tp_{A}≧4%.

5. The microparticles as claimed in any one of claims 2 to 4, in which the coating film A has a coating ratio (Tp_{A})—expressed as a dry weight % relative to the total mass of the coated microparticles—of less than or equal to 50%.

6. The microparticles as claimed in any one of claims 2 to 5, in which the coating film B has a coating ratio (Tp_{B})—expressed as a dry weight % relative to the total mass of the coated microparticles—of less than or equal to 50%.

7. The microparticles as claimed in any one of claims 2 to 6, in which the coating films A and B together represent not more than 50% of the dry weight relative to the total mass of the coated microparticles.

8. The microparticles as claimed in one of claims 2 to 7, in which the coating film A is an inner film directly in contact with the active principle particle and the coating film B is an outer film.

9. The microparticles as claimed in one of claims 2 to 7, in which the coating film B is an inner film directly in contact with the active principle particle and the coating film A is an outer film.

10. The microparticles as claimed in any one of claims 2 to 9, in which:
(A1) is chosen from the group comprising:
water-insoluble cellulose derivatives, preferably ethylcellulose and/or cellulose acetate,
acrylic derivatives, for example copolymers of (meth)acrylic acid and of alkyl (e.g. methyl) ester, copolymers of acrylic and methacrylic acid ester bearing at least one quaternary ammonium group (preferably at least one copolymer of alkyl (meth)acrylate and of trimethylammonioethyl methacrylate chloride) and more specifically the products sold under the brand names EUDRAGIT® RS and/or RL,
polyvinyl acetates,
and mixtures thereof;
(A2) is chosen from the group comprising:
nitrogenous (co)polymers, preferably from the group comprising polyacrylamides, poly-N-vinylamides, polyvinylpyrrolidones (PVP) and poly-N-vinyllactams;
water-soluble cellulose derivatives,
polyvinyl alcohols (PVA),
polyoxyethylenes (POE),
polyethylene glycols (PEG),
and mixtures thereof;
polyvinylpyrrolidone being particularly preferred;
(A3) is chosen from the group comprising:
cetyl alcohol esters
glycerol and esters thereof, preferably from the following subgroup: acetylated glycerides, glyceryl monostearate, glyceryl triacetate, glyceryl tributyrate,
phthalates, preferably from the following subgroup: dibutyl phthalate, diethyl phthalate, dimethyl phthalate, dioctyl phthalate,
citrates, preferably from the following subgroup: acetyl tributyl citrate, acetyl triethyl citrate, tributyl citrate, triethyl citrate,
sebacates, preferably from the following subgroup: diethyl sebacate, dibutyl sebacate,
adipates,
azelates,
benzoates,
plant oils,
fumarates, preferably diethyl fumarate,
malates, preferably diethyl malate,
oxalates, preferably diethyl oxalate,
succinates, preferably dibutyl succinate,
butyrates,
cetyl alcohol esters,
salicylic acid,
triacetin,
malonates, preferably diethyl malonate,
castor oil (this being particularly preferred),
and mixtures thereof;
(A4) is chosen from the group comprising:
anionic surfactants, preferably from the subgroup of alkali metal or alkaline-earth metal salts of fatty acids, stearic acid and/or oleic acid being preferred,
and/or nonionic surfactants, preferably from the following subgroup:
polyoxyethylenated oils, preferably hydrogenated polyoxyethylenated castor oil,
polyoxyethylene-polyoxypropylene copolymers,
polyoxyethylenated sorbitan esters,
polyoxyethylenated castor oil derivatives,
stearates, preferably calcium, magnesium, aluminum or zinc stearate,
stearylfumarates, preferably sodium stearylfumarate,
glyceryl behenates,
and mixtures thereof.

11. The microparticles as claimed in one of claims 2 to 10, in which the coating film A has the following quantitative weight percentage composition:
(A1) between 10 and 90 and preferably between 15 and 80,
(A2) between 5 and 50 and preferably between 10 and 40,
(A3) between 1 and 30 and preferably between 2 and 20,
(A4) between 0 and 20 and preferably between 0 and 15.

12. The microparticles as claimed in any one of claims 2 to 11, in which the hydrophilic polymer bearing groups that are ionized at neutral pH (B1) is chosen from the group comprising:
B1.a copolymers of (meth)acrylic acid and of alkyl (e.g. methyl) ester of (meth)acrylic acid (for example EUDRAGIT® S or L);
B1.b cellulose derivatives, preferably: cellulose acetates, cellulose phthalates, cellulose succinates and even more preferentially hydroxypropylmethylcellulose phthalates, hydroxypropylmethylcellulose acetates and hydroxypropylmethylcellulose succinates;
and mixtures thereof.

13. The microparticles as claimed in any one of claims 2 to 12, in which compound (B2) is chosen from the following group of products:
B2.a plant waxes taken alone or as mutual mixtures;
B2.b hydrogenated plant oils taken alone or as mutual mixtures;
B2.c mono- and/or di- and/or triesters of glycerol and of at least one fatty acid;
B2.d mixtures of monoesters, diesters and triesters of glycerol and of at least one fatty acid;
B2.e and mixtures thereof.

14. The microparticles as claimed in claim 13, in which compound (B2) is chosen from the following group of products: hydrogenated cottonseed oil, hydrogenated soybean oil, hydrogenated palm oil, glyceryl behenate, hydrogenated castor oil, tristearine, tripalmitine, trimyristine, yellow wax, hard fat or fat useful as suppository bases, anhydrous dairy fat, lanolin, glyceryl palmitostearate, glyceryl stearate, lauryl macrogolglycerides, cetyl alcohol, polyglyceryl diisostearate, diethylene glycol monostearate, ethylene glycol monostearate, omega-3 and any mixture thereof.

15. The microparticles as claimed in any one of claims 2 to 14, in which the weight ratio (B2)/(B1) is between 0.2 and 1.5 and preferably between 0.45 and 1.0.

16. The microparticles as claimed in any one of claims 2 to 15, in which the hydrophobic compound (B2) is chosen from products that are crystalline in the solid state and that have a melting point Tm(B2)≧40° C., preferably Tm(B2)≧50° C. and even more preferentially 50° C.≦Tm(B2)≦90° C.

17. The microparticles as claimed in any one of the preceding claims, in which the AP used belongs to at least one of the following families of active substances: agents for treating alcohol abuse, agents for treating Alzheimer's disease, anesthetics, agents for treating acromegaly, analgesics, antiasthmatic agents, agents for treating allergies, anticancer agents, antiinflammatories, anticoagulants and antithrombotic agents, hypolipemiants, anticonvulsants, antiepileptic agents, antidiabetic agents, antiemetic agents, antiglaucoma agents, antihistaminics, antiinfectious agents, antibiotics, antifungal agents, antiviral agents, antiparkinson agents, anticholinergic agents, antitussive agents, carbonic anhydrase inhibitors, cardiovascular agents, antiarrhythmic agents, vasodilators, antiangina agents, antihypertensives, vasoprotective agents, cholinesterase inhibitors, agents for treating central nervous system disorders, central nervous system stimulants, contraceptives, fertility promoters, labor inducers and inhibitors, agents for treating mucoviscidosis, dopamine receptor agonists, agents for treating endometriosis, agents for treating erectile dysfunction, agents for treating fertility, agents for treating gastrointestinal disorders, immunomodulators and immunosuppressants, agents for treating memory disorders, antimigraine agents, muscle relaxants, nucleoside analogs, agents for treating osteoporosis, parasympathomimetic agents, prostaglandins, psychotherapeutic agents, sedatives, hypnotics and tranquilizers, neuroleptic agents, anxiolytic agents, psychostimulants, antidepressants, dermatological treatment agents, steroids and hormones.

18. The microparticles as claimed in claim 17, in which:
examples of agents for treating acromegaly include: octreotide, laureotide and pegvisomant, and salts thereof, esters thereof, hydrates thereof, polymorphs thereof and isomers thereof;
examples of agents for treating alcohol abuse include: chlorazepate, chlordiazepoxide, diazepam, disulfuram, hydroxyzine, naltrexone, and salts thereof, esters thereof, hydrates thereof, polymorphs thereof and isomers thereof;
examples of anesthetics include: adrenalin, bupivacaine, chloroprocaine, desflurane, etidocaine, levobupivacaine, lidocaine, midazolam, propofol, ropivacaine, and salts thereof, esters thereof, hydrates thereof, polymorphs thereof and isomers thereof;
examples of analgesics include: acetaminophen, aspirin, bupivacaine, buprenorphine, butorphanol, celecoxib, clofenadol, choline, clonidine, codeine, diflunisal, dihydrocodeine, dihydroergotamine, dihydromorphine, ethylmorphine, etodolac, eletriptan, eptazocine, ergotamine, fentanyl, fenoprofen, hyaluronic acid, hydrocodone, hydromorphone, hylane, ibuprofen, indomethacin, ketorolac, ketotifen, levomethadone, levallorphan, levorphanol, lidocaine, mefenamic acid, meloxicam, meperidine, methadone, morphine, nabumetone, nalbuphine, nefopam, nalorphine, naloxone, naltrexone, naproxen, naratriptan, nefazodone, mormethadone, oxapozine, oxycodone, oxymorphone, pentazocine, pethidine, phenpyramide, piritramide, piroxicam, propoxyphen, refecoxib, rizatriptan, ketoprofen, sulindac, sumatriptan, tebacone, tilidine, tolmetine, tramadol, zolmitriptan, and salts thereof, esters thereof, hydrates thereof, polymorphs thereof and isomers thereof;
examples of antiasthmatic agents include: ablukast, azelastine, bunaprolast, cinalukast, cromitrile, cromolyne, enofelast, isambxole, ketotifen, levcromekaline, lodoxamide, montelukast, ontazolast, oxarbazole, oxatomide, piriprost potassium, pirolate, pobilukast, edamine, pranlukast, quazolast, repirinast, ritolukast, sulukast, tetrazolastmeglumine, tiaramide, tibenelast, tomelukast, tranilast, verlukast, verofylline, zarirlukast, and salts thereof, esters thereof, hydrates thereof, polymorphs thereof and isomers thereof;
examples of anticancer agents include: adriamycin, aldesleukin, allopurinol, altretamine, amifostine, anastrozole, asparaginase, betamethasone, bexaroten, bicalutamide, bleomycin, busulfan, capecitabine, carboplatin, carmustine, chlorambucil, cisplatin, cladribine, conjugated estrogen, cortisone, cyclophosphamide, cytarabine, dacarbazine, daunorubicin, dactinomycin, denileukin, dexamethasone, discodermolide, docetaxel, doxorubicin, eloposidem, epirubicin, epoetin, epothilones, estramustine, esterified estrogen, ethynyl-estradiol, etoposide, exemestane, flavopirdol, fluconazole, fludarabine, fluorouracil, flutamide, floxuridine, gemcitabine, gemtuzumab, goserelin, hexamethylmelamine, hydrocortisone, hydroxyurea, idarubicin, ifosfamide, interferon, irinotecan, lemiposide, letrozole, leuprolide, levamisole, levothyroxine, lomustine, mechlorethamine, melphalan, mercaptopurine, megestrol, methotrexate, methylprednisolone, methyltestosterone, mithramycin, mitomycin, mitotane, mitoxantrone, mitozolomide, mutamycin, nilutamide, paclitaxel, pamidronate, pegaspargase, pentostatin, plicamycin, porfimer, prednisolone, procarbazine, rituximab, sargramostim, semustine, streptozocin, tamoxifen, temozolamide, teniposide, testolactone, thioguanine, thiotepa, tomudex, topotecan, toremifen, trastumuzab, tretinoin, semustine, streptozolocin, valrubicin, verteprofin, vinblastine, vincristine, vindesine, vinorelbine, and salts thereof, esters thereof, hydrates thereof, polymorphs thereof and isomers thereof;
examples of anticoagulants and antithrombotic agents include: warfarin, dalteparine, heparine, tinzaparin, enoxaparin, danaparoid, abciximab, alprostadil, altiplase, anagralide, anistreplase, argatroban, ataprost, betaprost, camonagrel, cilostazol, clinprost, clopidogrel, cloricromen, dermatan, desirudine, domitroban, drotaverine, epoprostenol, eptifibatide, fradafiban, gabexate, iloprost, isbogrel, lamifiban, lamoteplase, lefradafiban, lepirudin, levosimendan, lexipafant, melagatran, nafagrel, nafamostsat, nizofenone, orbifiban, ozagrel, pamicogrel, parnaparin, quinobendan, reteplase, sarpogralate, satigrel, silteplase, simendan, ticlopidine, vapiprost, tirofiban, xemilofiban, Y20811, and salts thereof, esters thereof, hydrates thereof, polymorphs thereof and isomers thereof;
examples of anticonvulsants include: carbamazepine, clonazepam, clorazepine, diazepam, divalproex, ethosuximide, ethotion, felbamate, fosphenyloin, gabapentine, lamotrigine, levetiracetam, lorazepam, mephenyloin, mephobarbital, metharbital, methsuximide, oxcarbazepine, phenobarbital, phenyloin, primidone, tiagabine, topiramate, valproic acid, vigabatrin, zonisamide, and salts thereof, esters thereof, hydrates thereof, polymorphs thereof and isomers thereof;
examples of antidiabetic agents include: acarbose, acetohexamide, carbutamide, chlorpropamide, epalrestat, glibornuride, gliclazide, glimepiride, glipizide, gliquidone, glisoxepide, glyburide, glyhexamide, metformin, miglitol, nateglinide, orlistat, phenbutamide, pioglitazone, repaglinide, rosiglitazone, tolazamide, tolbutamide, tolcyclamide, tolrestat, troglitazone, voglibose, and salts thereof, esters thereof, hydrates thereof, polymorphs thereof and isomers thereof;
examples of antiemetic agents include: alprazolam, benzquinamide, benztropine, betahistine, chlorpromazine, dexamethasone, difenidol, dimenhydrinate, diphenhydramine, dolasetron, domperidone, dronabinol, droperidol, granisetron, haloperidol, lorazepam, meclizine, methylprednisolone, metoclopramide, ondansetron, perphenazine, prochlorperazine, promethazine, scopolamine, tributin, triethylperazine, triflupromazine, trimethobenzamide, tropisetron, and salts thereof, esters thereof, hydrates thereof, polymorphs thereof and isomers thereof;
examples of antiglaucoma agents include: alprenoxime, dapiprazole, dipivefrin, latanoprost, naboctate, pimabine, and salts thereof, esters thereof, hydrates thereof, polymorphs thereof and isomers thereof;
examples of antihistaminics include: acepromazine, acrivastine, activastine, albuterol, alimemazine, antazoline, azelastine, bitolterol, amlexanox, benzydamine, brompheniramine, cetirizine, chlorpheniramine, cimetidine, cinnarizine, clemastine, clofedanol, cycloheptazine, cyproheptadine, diclofenac, difencloxazine, diphenhydramine, dotarizine, ephedrine, epinastine, epinephrine, ethylnorepinephrine, etybenzatropine, fenpentadiol, fenpoterol, fexofenadine, flurbiprofen, hydroxyzine, isoetharine, isoproterenol, ipratropium bromide, ketorolac, levocetirizine, levomepromazine, loratidine, mequitazine, metaproterenol, niaprazine, oxatomide, oxomemazine, phenylephrine, phenylpropanolamine, pirbuterol, promethazine, pseudoephedrine, pyrilamine, salmeterol, terbutaline, terfenadine, tranilast, xanthine derivatives, xylometazoline, and salts thereof, esters thereof, hydrates thereof, polymorphs thereof and isomers thereof;
examples of antiinfectious agents, especially antibiotics, antifungal agents and antiviral agents, include: abacavir, aciclovir, albendazole, amantadine, amphotericin, amikacin, aminosalicylic acid, amoxycillin, ampicillin, amprenavir, atovaquine, azithromycin, aztreonam, carbenicillin, cefaclor, cefadroxil, cefamandole, cefazolin, cefdinir, cefepime, cefexime, cefoperazone, cefotaxime, cefotitam, cefoperazone, cefoxitine, cefpodoxine, cefprozil, ceftazidime, ceftibuten, ceftizoxime, ceftriaxone, cefuroxirme, cephalexine, chloroquine, cidofovir, cilastatin, ciprofloxacin, clarithromycin, clavulanic acid, clindamycin, colistimethate, dalfopristin, dapsone, daunorubicin, delavirdine, demeclocycline, didanosine, doxycycline, doxorubicin, efavirenz, enoxacin, erythromycin, ethambutol, ethionamide, famcyclovir, fluconazole, flucytocine, foscarnet, fosfomycin, ganciclovir, gatifloxacin, griseofulvin, hydroxychloroquine, imipenem, indinavir, interferon, isoniazide, itraconazole, ivermectil, ketoconazole, lamivudin, levofloxacin, linizolide, lomefloxacin, loracarbef, mebendazole, mefloquine, meropenem, methanamine, metronidazole, minocycline, moxefloxacin, naldixic acid, nelfinavir, neomycin, nevirapine, nitorfurantoin, norfloxacin, ofloxacin, oseltamivir, oxytetracycline, palivizumab, penicillin, perfloxacin, piperacillin, praziquantel, pyrazinamide, pyrimethamine, quinidine, quinupristine, retonavir, ribavirin, rifabutin, rifampicin, rimantadine, saquinavir, sparfloxacin, stavudin, streptomycin, sulfamethoxazole, tetramycin, terbinafine, tetracycline, ticarcillin, thiabendazole, tobramycin, trimethoprim, trimetraxate, troleandomycin, trovafloxacin, valaciclovir, vancomycin, zalcitabine, zanamivir, zidovudine, and salts thereof, esters thereof, hydrates thereof, polymorphs thereof and isomers thereof;
examples of antiparkinson agents include: amantadine, adrogolide, altinicline, benzatropine, biperiden, brasofensine, bromocriptine, budipine, cabergoline, CHF-1301, dihydrexidine, entacapone, etilevodopa, idazoxane, iometopane, lazabemide, melevodopa, carbidopa, levodopa, mofegiline, moxiraprine, pergolide, pramipexole, quinelorane, rasagiline, ropinirole, seligiline, talipexole, tolcapone, trihexyphenidyl, and salts thereof, esters thereof, hydrates thereof, polymorphs thereof and isomers thereof;
examples of antirheumatic agents include: azathiprine, betamethasone, celecoxib, cyclosporine, diclofenac, hydroxychloroquine, indomethacin, infliximab, mercaptobutanedioic acid, methylprednisolone, naproxen, penicillamine, piroxicam, prednisolone, sulfasalazine, and salts thereof, esters thereof, hydrates thereof, polymorphs thereof and isomers thereof;
examples of anti-platelet-aggregating agents include: abciximab, anagrelide, aspirin, cilostazol, clopidogrel, dipyridamole, epoprostenol, eptifibatide, ticlopidine, tinofiban, and salts thereof, esters thereof, hydrates thereof, polymorphs thereof and isomers thereof;
examples of antispasmodic and anticholinergic agents include: aspirin, atropine, diclofenac, hyoscyamine, mesoprostol, methocarbamol, phenobarbital, scopolamine, and salts thereof, esters thereof, hydrates thereof, polymorphs thereof and isomers thereof;
examples of antitussive agents include: acetaminophen, acrivastine, albuterol, benzonatate, beractant, brompheniramine, caffeine, calfactant, carbetapentane, chlorpheniramine, codeine, colfuscerine, dextromethorpham, dornase alpha, doxylamine, epinephrine, fexofenadine, guaphenesin, ipratropium, levalbuterol, metaproterenol, montelukast, pentoxyphilline, phenylephrine, phenylpropanolamine, pirbuterol, poractant alpha, pseudoephedrine, pyrilamine, salbuterol, salmeterol, terbutaline, theophylline, zafirlukast, zileuton, and salts thereof, esters thereof, hydrates thereof, polymorphs thereof and isomers thereof;
examples of carbonic anhydrase inhibitors include: acetazolamide, dichlorphenamide, dorzolamide, methazolamide, sezolamide, and salts thereof, esters thereof, hydrates thereof, polymorphs thereof and isomers thereof;
examples of cardiovascular agents, especially hypolipemiants, antiarrhythmic agents, vasodilators, antiangina agents, antihypertensives and vasoprotective agents, include: abciximab, acebutolol, activase, adenosine, adrenaline, amidarone, amiloride, amlodipine, amyl nitrate, atenolol, atorvastatin, benzepril, bepiridil, betaxalol, bisoprolol, candesartan, captopril, cartenolol, carvedilol, cerivastatin, chlorthalidone, chlorthiazole, clofibrate, clonidine, colestipol, colosevelam, digoxin, diltiazem, disopyramide, dobutamine, dofetilide, doxazosine, enalapril, epoprostenol, eprosartan, esmolol, ethacrynate, erythrityl, felodipine, fenoidapam, fosinopril, flecamide, fluorosemide, fluvastatin, gemfibrozil, hydrochlorthiazide, hydroflumethazine, ibutilide, indapamide, isosorbide, irbesartan, labetolol, lacidipine, lisinopril, losartan, lovastatin, mecamylamine, metoprolol, metaminol, metazolone, methylchlothiazide, methyldopa, metyrosine, mexiletine, midrodine, milrinone, moexipril, nadolol, niacin, nicardipine, nicorandil, nifedipine, nimodipine, nisoldipine, nitroglycerine, phenoxybenzamine, perindopril, polythiazide, pravastatin, prazosin, procainamide, propafenone, propranolol, quanfacine, quinapril, quinidine, ranipril, reteplase, simvastatin, sotalol, spironolactone, streptokinase, telmisartan, terazosin, timolol, tocainamide, torsemide, trandolapril, triamteren, trapidil, valsartan, and salts thereof, esters thereof, hydrates thereof, polymorphs thereof and isomers thereof;
examples of vasodilators include: adenosine, alverine, caffeine, dihydroergocornine, enalapril, enoximone, iloprost, kalleone, lidoflazine, nicardipine, nimodipine, nicotinic acid, papaverine, pilocarpine, salbutamol, theophylline, trandolapril, uradipil, vincamine, and salts thereof, esters thereof, hydrates thereof, polymorphs thereof and isomers thereof;
examples of cholinesterase inhibitors include: donepezil, edrophonium, neostigmine, pyridostigmine, rivastigmine, tacrine, and salts thereof, esters thereof, hydrates thereof, polymorphs thereof and isomers thereof;
examples of central nervous system stimulants include: caffeine, doxapram, dexoamphetamine, donepezil, edorphonium, methamphetamine, methylphenidate, modafinil, neostigmine, pemoline, phentermine, pyridostigmine, rivastigmine, tacrine, and salts thereof, esters thereof, hydrates thereof, polymorphs thereof and isomers thereof;
examples of contraceptives include: desogestral, ethinyl-estradiol, ethynodiol, levonorgestrel, medroxyprogesterone, mestranol, norgestimate, norethindrone, norgestrel, and salts thereof, esters thereof, hydrates thereof, polymorphs thereof and isomers thereof;
examples of agents for treating mucoviscidosis include: domase alpha, pancrelipase, tobramycin, and salts thereof, esters thereof, hydrates thereof, polymorphs thereof and isomers thereof;
examples of dopamine receptor agonists include: amantadine, cabergoline, fenoldopam, pergolide, pramipezal, ropinirole, and salts thereof, esters thereof, hydrates thereof, polymorphs thereof and isomers thereof;
examples of agents for treating endometriosis include: danazol, goserelin, leuprolide, nafarelin, norethindrone, and salts thereof, esters thereof, hydrates thereof, polymorphs thereof and isomers thereof;
examples of agents for treating erectile dysfunction include: alprostadil, sildenafil, yohimbine, and salts thereof, esters thereof, hydrates thereof, polymorphs thereof and isomers thereof;
examples of agents for treating fertility include: citrorelix, clomiphen, follitropine, ganirelix, gonadotropin, menotropin, progesterone, urofollitropin, and salts thereof, esters thereof, hydrates thereof, polymorphs thereof and isomers thereof;
examples of agents for treating gastrointestinal disorder include: alosetron, bisacodyl, bismuth subsalicylate, celecoxib, cimetidine, difoxine, dipheoxylate, docusate, esomeprazole, famotidine, glycopyrrolate, infliximab, lansoprazole, loperamide, metaclopramide, nizatidine, omeprazole, pantoprazole, rabeprazole, ranitidine, simethicone, sucralfate, and salts thereof, esters thereof, hydrates thereof, polymorphs thereof and isomers thereof;
examples of immunomodulators and immunosuppressants include: azathioprine, ceftizoxine, cyclosporine, daclizumab, glatiramer, immunoglobulin, interferon, leflunomide, levamisol, mycophenolate, phthalidomide, ribavirine, sirolimus, and salts thereof, esters thereof, hydrates thereof, polymorphs thereof and isomers thereof;
examples of agents for treating Alzheimer's disease include: CP 118954, donepezil, galanthamine, metrifonate, revastigmine, tacrine, TAK-147, and salts thereof, esters thereof, hydrates thereof, polymorphs thereof and isomers thereof;
examples of antimigraine agents include: acetaminophen, dihydroergotamine, divalproex, ergotamine, propranolol, risatriptan, sumatriptan, trimetrexate, and salts thereof, esters thereof, hydrates thereof, polymorphs thereof and isomers thereof;
examples of muscle relaxants include: alcuronium chloride, azapropazone, atracurium, baclofen, carisoprodol, quinine derivatives, chloromezanone, chlorophenesincarbamate, chlorozoxazone, cyclobenzaprine, dantrolen, decamethonium bromide, dimethyltubocurarinium chloride, doxacurium, fenyramidol, gallamine triethiodide, guaiphenesin, hexafluorenium bromide, hexacarbacholine bromide, memantin, mephenesin, meprobamate, metamisol, metaxalone, methocarbamol, mivacurium, orphenadrine, pancuronium, phenazone, phenprobamate, pipecuronium, rapacuronium, rocuronium, succinylcholine, suxamethonium chloride, tetrazepam, tizanidine, tubocurarine chloride, tybamate, vecuronium, and salts thereof, esters thereof, hydrates thereof, polymorphs thereof and isomers thereof;
examples of nucleoside analogs include: abacavir, aciclovir, didanosine, gamciclovir, gemcitabine, lamivudine, ribavirin, stavudine, zalcitabine, and salts thereof, esters thereof, hydrates thereof, polymorphs thereof and isomers thereof;
examples of agents for treating osteoporosis include: alendronate, calcitonin, estradiol, estropipate, medroxyprogesterone, norethindrone, norgestimate, pamidronate, raloxifen, risdronate, zoledronate, and salts thereof, esters thereof, hydrates thereof, polymorphs thereof and isomers thereof;
examples of parasympathomimetic agents include: bethanechol, piperidine, edrophonium, glycopyrolate, hyoscyamine, pilocarpine, tacrine, yohimbine, and salts thereof, esters thereof, hydrates thereof, polymorphs thereof and isomers thereof;
examples of prostaglandins include: alprostadil, epoprostenol, misoprostol, and salts thereof, esters thereof, hydrates thereof, polymorphs thereof and isomers thereof;
examples of psychotherapeutic agents include: acetophenazine, alentemol, alpertine, alprazolam, amitriptyline, apriprazole, azaperone, batelapine, befipiride, benperidol, benzindopyrine, bimithil, biriperone, brofoxine, bromperidol, broniperidol, bupropione, buspirone, butaclamol, butaperazine, butaperazin, carphenazine, carvotroline, cericlamine, chlorazepine, chlordiazepoxide, chlorpromazine, chlorprothixen, cinperen, cintriamide, citalopram, clomacran, clonazepam, clopenthixol, clopimozide, clopipazan, cloroperone, clothiapine, clothixamide, clozapine, cyclophenazine, dapiprazole, dapoxetine, desipramine, divalproex, dipyridamole, doxepin, droperidol, duloxetine, eltoprazine, eptipirone, etazolate, fenimide, flibanserine, flucindole, flumezapine, fluoxetine, fluphenazine, fluspiperone, fluspirilen, flutroline, fluvoxamine, gepirone, gevotroline, halopemide, haloperidol, hydroxyzine, hydroxynortriptyline, iloperidone, imidoline, lamotrigine, loxapine, enperone, mazapertine, mephobarbital, meprobamate, mesoridazine, mesoridazine, milnacipran, mirtazepine, metiapine, milenperone, milipertine, molindone, nafadotride, naranol, nefazodone, neflumozide, ocaperidone, odapipam, olanzapine, oxethiazine, oxiperomide, pagoclone, paliperidone, paroxiten, penfluridol, pentiapine, perphenazine, phenelzine, pimozide, pinoxepin, pipamperone, piperacetazine, pipotiazine, piquindone, pirlindole, pivagabine, pramipexole, prochlorperazine, promazine, quetiapine, reboxetine, remoxipride, risperidone, rimcazole, robolzotan, selegiline, seperidol, sertraline, sertindole, seteptiline, setoperone, spiperone, sunipitrone, tepirindole, thioridazine, thiothixen, tiapride, tioperidone, tiospirone, topiramate, tranylcypromine, trifluoperazine, trifluperidol, triflupromazine, trimipramine, venlafaxine, ziprasidone, and salts thereof, esters thereof, hydrates thereof, polymorphs thereof and isomers thereof;
examples of sedatives, hypnotics and tranquilizers include: bromazepam, buspirone, clazolam, clobazam, chlorazepate, diazepam, demoxepam, dexmedetomidine, diphenyhydramine, doxylamine, enciprazine, estrazolam, hydroxyzine, ketazolam, lorazatone, lorazepam, loxapine, medazepam, meperidine, methobarbital, midazolam, nabilone, nisobamate, oxazepam, pentobarbital, promethazine, propofol, triazolam, zaleplon, zolpidem, and salts thereof, esters thereof, hydrates thereof, polymorphs thereof and isomers thereof;
examples of dermatological treatment agents include: acitretin, alclometasone, alitretinoin, betamethasone, calciprotrine, chlorhexidine, clobetasol, clocortolone, clotriamozole, collagenase, cyclosporine, desonide, difluorosone, doxepine, eflomithine, finasteride, fluocinolone, flurandrenolide, fluticasone, halobetasol, hydrochloroquine, hydroquinone, hydroxyzine, ketoconazole, mafenide, malathion, menobenzone, neostigmine, nystatin, podofilox, povidone, tazoroten, tretinoin, and salts thereof, esters thereof, hydrates thereof, polymorphs thereof and isomers thereof;
examples of steroids and hormones include: alclometasone, betamethasone, calcitonine, citrorelix, clobetasol, clocortolone, cortisones, danazol, desmopressin, desonide, desogestrel, desoximetasone, dexamethasone, diflorasone, estradiol, estrogens, estropipate, ethynilestradiol, fluocinolone, flurandrenolide, fluticasone, glucagon, gonadotropin, goserelin, halobetasol, hydrocortisone, leuprolide, levonorgestrel, levothyroxine, medroxyprogesterone, menotropins, methylprednisolone, methyltestosterone, mometasone, naferelin, norditropin, norethindrone, norgestrel, octreolide, oxandrolone, oxymetholone, polytropin, prednicarbate, prednisolone, progesterone, sermorelin, somatropin, stanozolol, testosterone, urofollitropin, and salts thereof, esters thereof, hydrates thereof, polymorphs thereof and isomers thereof.

19. A pharmaceutical, veterinary or dietetic formulation, characterized in that it comprises a plurality of coated microparticles as claimed in any one of the preceding claims, advantageously at least 500, preferably from 1000 to 1 000 000 and even more preferentially from 5000 to 500 000 microparticles.

20. A pharmaceutical, veterinary or dietetic formulation, characterized in that it comprises coated microparticles as claimed in any one of claims 1 to 18 and in that it is in a pharmaceutical form chosen from the group comprising: tablets, powders, suspensions, syrups, powders for suspension to be reconstituted, or gel capsules.

21. The formulation as claimed in claim 19 or 20, also comprising at least one active principle in an immediate-release form.

22. The formulation as claimed in claim 21, in which at least one active principle in an immediate-release form is the same as an active principle contained in at least some of the microparticles.

23. The formulation as claimed in any one of claims 19 to 22, comprising a plurality of populations of microparticles, said populations differing from each other by their lag time and/or by their triggering pH and/or by their rate of release and/or by the active principle they contain.

24. The use of the coated microparticles as claimed in any one of claims 1 to 18, for the preparation of pharmaceutical, veterinary or dietetic formulations as claimed in any one of claims 19 to 23.
